(19) United States
(12) Patent Application Publication
(10) Pub. No.: US 2024/0071566 A1
(43) Pub. Date: Feb. 29, 2024

(54) MACHINE PERCEPTION NANOSENSOR ARRAYS AND COMPUTATIONAL MODELS FOR IDENTIFICATION OF SPECTRAL RESPONSE SIGNATURES

(71) Applicants: Memorial Sloan- Kettering Cancer Center, New York, NY (US); Sloan-Kettering Institute For Cancer Research, New York, NY (US); Memorial Hospital for Cancer and Allied Diseases, New York, NY (US); Lehigh Univer, Bethlehem, PA (US); University of Maryland, College Park, MD (US); National Institue of Standards and Technolgy, Gaitherburg, MD (US)

(72) Inventors: Daniel A. HELLER, New York, NY (US); Mijin KIM, New York, NY (US); Yoona YANG, Bethlehem, PA (US); Yuhuang WANG, Laurel, MD (US); Anand JAGOTA, Bethlehem, PA (US); Ming ZHENG, Rockville, MD (US)

(21) Appl. No.: 18/262,300

(22) PCT Filed: Jan. 20, 2022
(86) PCT No.: PCT/US22/13190

§ 371 Date: Jul. 20, 2023

Related U.S. Application Data
(60) Provisional application No. 63/140,136, filed on Jan. 21, 2021.
(60) Provisional application No. 63/194,722, filed on May 28, 2021.

(51) Int. Cl. G16B 20/20; G06N 20/10; G06N 20/20; G16B 25/00; G16B 40/20

(52) U.S. Cl. CPC G16B 20/20; G06N 20/10; G06N 20/20; G16B 25/00; G16B 40/20

(57) ABSTRACT

Disclosed are approaches to acquiring a “disease fingerprint” from biosamples by collecting large data sets of physicochemical interactions to a sensor array composed of organic color center-modified carbon nanotubes. Array responses from subjects may be used to train and validate machine learning models to differentiate diseases and healthy individuals. The trained learning models may be used to subsequently classify patients based on nanosensor array emission data.

### CROSS-REFERENCE TO RELATED APPLICATIONS

This application is the U.S. National Stage Entry of International Application No. PCT/US2022/013190, filed Jan. 20, 2022, which claims the benefit of and priority to U.S. Provisional Patent Application No. 63/140,136 filed Jan. 21, 2021, and U.S. Provisional Patent Application No. 63/194,722, filed May 28, 2021, the entirety of each of which is incorporated herein by reference.

### STATEMENT REGARDING FEDERALLY SPONSORED RESEARCH

This invention was made with government support under Grant R01CA215719 awarded by the National Cancer Institute. The government has certain rights in the invention.

### BACKGROUND

Medical conditions are often difficult to reliably detect, at least in a non-invasive or minimally invasive manner. Major factors limiting precise screening using, for example, serum biomarkers include patient heterogeneity and low specificity resulting from few established molecular markers. Known biomarkers may not represent a complete disease state or may be present in many other diseases. Many serum biomarkers in clinical practice thus only provide incremental value for treatment options, and often do not reduce the screening cost for patients.

### SUMMARY

Various embodiments relate to a method comprising: receiving, by a computing system, emission data corresponding to fluorescence spectral responses of nanosensor arrays in contact with a plurality of biological samples collected from a cohort of subjects, the cohort of subjects including subjects with a medical condition and subjects without the medical condition, each nanosensor array comprising a semiconducting single-walled carbon nanotubes (SWCNT) that is (i) covalently functionalized and (ii) encapsulated by a nucleic acid; generating, by the computing system, based on the emission data, a dataset comprising a plurality of spectral feature changes caused by the biological samples, the spectral feature changes corresponding to intensity and wavelength of emissions from the nanosensor array in response to excitation by coherent light from a light source; training, by the computing system, a machine learning model based on the dataset and on clinical data corresponding to the medical condition for each subject in the cohort of subjects, wherein the machine learning model comprises at least one of logistic regression, decision tree, artificial neural networks (ANN), random forest, or support vector machine (SVM), wherein the machine learning model is configured to receive emission data and provide a classification corresponding to the medical condition; and providing, by the computing system, the machine learning model for classification of the medical condition in a patient based on spectral responses of nanosensor arrays in contact with a patient sample, wherein providing the machine learning model comprises at least one of storing the machine learning model in a non-volatile computer-readable storage medium of the computing system or transmitting the machine learning model to a second computing system.

In various embodiments, the spectral feature changes correspond to a plurality of an intensity of an E₁₁ peak (int), an intensity of an E₁₁-peak (int*), a wavelength of the E₁₁ peak (wl), and a wavelength of the and E₁₁-peak (wl*).

In various embodiments, the SWCNTs are functionalized by organic color centers (OCCs).

In various embodiments, the OCCs comprise an aryl functional group selected from the group consisting of 4-N,N-diethylamino (-4-N(C₂H₅)₂), 3,4,5-trifluoro (-3,4,5-F₃), or 3-fluoro-4-carboxy (-3-F-4-CO₂H).

In various embodiments, the SWCNTs are encapsulated by a single-strand deoxyribonucleic acid (ssDNA).

In various embodiments, the ssDNA comprises a sequence selected from a group consisting of CTTC₃TTC, (TAT)₄, or (GT)₁₅.

In various embodiments, the nanosensor arrays comprise OCC-functionalized, ssDNA-encapsulated SWCNTs selected from a group consisting of NEt₂*CTTC₃TTC, NEt₂*(TAT)₄, NEt₂*(GT)₁₅, 3F*CTTC₃TTC, 3F*(TAT)₄, 3F*(AT)₁₅, 3F*(GT)₁₅, F—CO₂H*CTTC₃TTC, F—CO₂H*(AT)₁₅, or F—CO₂H*(GT)₁₅, where NEt₂ represents 4-N,N-diethylamino, 3F represents F—CO₂H 3,4,5-trifluoro, and F—CO₂H represents 3-fluoro-4-carboxy aryl OCCs.

In various embodiments, the machine learning model is an SVM model trained by spectral responses of a plurality of OCC-DNA SWCNTs.

In various embodiments, the plurality of OCC-DNA SWCNTs comprise at least one OCC-DNA SWCNT selected from a group consisting of NEt₂*CTTC₃TTC, NEt₂*(TAT)₄, 3F*(TAT)₄, 3F*(AT)₁₅, or 3F*(GT)₁₅, where NEt₂ represents 4-N,N-diethylamino, 3F represents F—CO₂H 3,4,5-trifluoro, and F—CO₂H represents 3-fluoro-4-carboxy aryl OCCs.

In various embodiments, the method may comprise: receiving, by the computing system, emission data corresponding to fluorescence spectral responses of a nanosensor array in contact with a biological sample of a patient; and processing, by the computing system, the emission data using the machine learning to obtain a classification corresponding to the medical condition in the patient.

In various embodiments, the method may comprise administering a treatment to the patient based on the classification.

In various embodiments, the biological sample of the patient is a serum sample from the patient.

In various embodiments, the coherent light used for excitation has a wavelength bandwidth centered at 575 nanometers (nm).

In various embodiments, the method may comprise synthesizing the nanosensor arrays.

In various embodiments, synthesizing the nanosensor arrays may comprise introducing sp³ defects to (6,5) SWCNTs via diazonium chemistry and encapsulating the SWCNTs with a library ssDNA to solubilize the nanosensors in biofluids.

In various embodiments, the biological samples comprise sera of subjects in the cohort of subjects.

Various embodiments relate to a method comprising: receiving, by a computing system, emission data corresponding to fluorescence spectral responses of a nanosensor array in contact with a biological sample of the patient, the nanosensor array comprising a semiconducting single-walled carbon nanotubes (SWCNT) that is (i) covalently functionalized and (ii) encapsulated by a nucleic acid; and processing, by the computing system, the emission data using a machine learning model to obtain a classification corresponding to a medical condition in the patient, the machine learning model configured to provide the classification based on emission data corresponding to biological sample of the patient, the machine learning model having been trained based on reference emission data and clinical data corresponding to the medical condition for each subject in a cohort of subjects, the reference emission data corresponding to fluorescence spectral responses of nanosensor arrays in contact with a plurality of biological samples collected from the cohort of subjects, the cohort of subjects including subjects with a medical condition and subjects without the medical condition, the emission data having been used to generate a training dataset comprising a plurality of spectral feature changes caused by the biological samples, the spectral feature changes corresponding to intensity and wavelength of emissions from the nanosensor array in response to excitation by coherent light from a light source, wherein the machine learning model comprises at least one of logistic regression, decision tree, artificial neural networks (ANN), random forest, or support vector machine (SVM).

In various embodiments, the method may comprise administering a treatment to the patient based on the classification.

In various embodiments, the SWCNTs are functionalized by organic color centers (OCCs), and the SWCNTs are encapsulated by a single-strand deoxyribonucleic acid (ssDNA).

In various embodiments, the OCCs comprise an aryl functional group selected from the group consisting of 4-N,N-diethylamino (-4-N(C₂H₅)₂), 3,4,5-trifluoro (-3,4,5-F₃), or 3-fluoro-4-carboxy (-3-F-4-CO₂H), and the ssDNA comprises a sequence selected from a group consisting of CTTC₃TTC, (TAT)₄, or (GT)₁₅.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A: Example system for implementing disclosed approaches, according to various potential embodiments.

FIG. 1B: Example process for obtaining and using the disclosed nanosensor arrays and computational models, according to various potential embodiments.

FIGS. 2A and 2B: an OCC-DNA nanosensor array, according to various potential embodiments. (2A) Molecular model of an OCC-DNA nanosensor element. Shown is a ss(GT)₁₅ DNA-wrapped (6,5)-SWCNT with 3,4,5-trifluoroaryl OCC. (2B) Construction of an OCC-DNA nanosensor array from OCC and ssDNA components.

FIGS. 3A-3D: Spectroscopic responses of OCC-DNA sensors to patient serum samples. (3A), Representative fluorescence spectra of the ss(GT)₁₅ wrapped 3,4,5-trifluoroaryl OCC sensor, 3F*(GT)₁₅, in PBS (gray), 20 v/v % serum from an HGSOC patient (orange) and serum from a healthy individual (blue). (3B) Spectral responses of the 3F*(GT)₁₅ sensor to cancer and healthy individuals' serum samples. Four spectral parameters—intensity and wavelength of the E₁₁ and E₁₁-peaks (int, int*, wl, and wl*) were extracted from fluorescence spectra of four serum samples for each group. Data points represent the mean value of the spectroscopic variables. Each sample was measured in triplicate. Horizontal lines denote the median. (3C) E₁₁ intensity change (dint) of each OCC-DNA sensor in response to 215 serum samples from HGSOC and other disease patients, as well as healthy individuals at 2-hour incubation. (3D) Principal component analysis (PCA) of sensor responses to HGSOC (orange), other diseases (light blue), and healthy samples (blue).

FIGS. 4A-4G: Optimization of machine learning algorithms for HGSOC classification. (4A), Comparison of F-scores of HGSOC identification with artificial neural network (ANN), random forest (RF), and SVM models, using sensor data collected with different serum incubation times. (4B), Distribution of F-scores obtained using data with different numbers of spectral variables: 2 variables (Δwl+Δint) vs. 4 variables (dwl+dwl*+dint+dint*) vs. 6 variables (dwl+dwl*+dint+dint*+Δwl+Δint). (4C) F-scores obtained with different numbers of OCC-DNA nanosensor types, via SVM. (4D) Sensitivity at 98% specificity obtained with varying β in F_{β} scoring via SVM. The line connects the median of sensitivities for the optimized nanosensor arrays. (4E) Best ROC curves for binary classification of HGSOC, showing both cross-validated training set (CV) and test/validation set (Test). The shaded area is the standard deviation of 10-fold validation. (4F) ROC curves of HGSOC classification using individual serum biomarkers (CA125: blue, HE4: orange, YKL40: gray) and logistic regression of their combination (black). (4G) PCA plot of three disease states, HGSOC (orange), other diseases (light blue), and healthy patients (blue), calculated using conventional serum measurements of CA125, HE4, and YKL40 levels from 215 patient sera.

FIGS. 5A-5I: Known serum biomarkers make up part of the disease fingerprint in the nanosensor array platform. (5A-5D) Representative spectral response of OCC-DNA in 20% FBS at increasing concentration of (5A) CA125, (5B, 5C) HE4, and (5D) YKL40. (5E) Feature importance analysis of the binary SVM model. 5F, ROC curves of binary biomarker classification (normal vs. above clinical reference) using SVM of the OCC-DNA sensor responses. (5G) F-score ranges of SVM classifications of HGSOC biomarkers or disease state. Lines in each box indicates the median. (5H) R-squared ranges of biomarker SVR. (5I) Serum CA125 levels predicted by SVR against immunoassay results. The prediction models were trained by the fluorescence response of NEt₂*(TAT)₄, 3F*(TAT)₄, and 3F*(AT)₁₅. The highlighted squares classify normal (<50 U/mL, blue) and high CA125 (>250 U/mL, red) groups.

FIG. 6: Fluorescence spectra of OCC-DNA complexes in PBS. The excitation wavelength was 575 nm.

FIGS. 7A and 7B: Spectral responses of OCC-DNAs to a small set of HGSOC and benign serum samples. Four spectral parameters—intensity and wavelength changes of the E₁₁ and E₁₁-peaks—were extracted from fluorescence spectra of four serum samples in each group. Each sample was measured in triplicate. Horizontal lines denote the median. Six OCC-DNA nanosensors, with p-values of the spectroscopic features lower than 0.10, were selected for the sensor array.

FIG. 8: Frequency distribution of variation in E₁₁ and E₁₁-wavelength shifts. The variation was calculated as the difference between the center wavelength of each measurement from the average of the triplicate measurements.

FIGS. 9A-9D: Spectral responses of the nanosensor array to training and validation sets of patient serum samples (Nₛₐ=215). Four spectral parameters, a, dint, b, dint*, c, dwl, and d, dwl*, were extracted from fluorescence spectra of the sensor array after 2-hour serum incubation. Each sample was measured in triplicate.

FIG. 10: Averaged F-scores of optimized machine learning models with 10-fold validation. The classification was divided as HGSOC versus other gynecologic diseases and benign groups. The blue line is the logarithmic regression of the median F-score.

FIGS. 11A-11C: Assessment of medications as potential interferents to nanosensor prediction. a, Fraction of medication dose for HGSOC and other disease patients. b, Chronic conditions, and prevalence thereof, in patients measured in this study. Comorbidity was identified based on the patients' medication information. c, Anti-cancer drugs or prescription drugs whose occurrence differed by 0.1 or higher between HGSOC and other disease groups.

FIGS. 12A-12C: Serum levels of known ovarian cancer biomarkers in the model study population. a, CA125, b, HE4, and c, YKL40. The serum protein levels were quantified by automated immunoassay. Dotted lines indicate the clinical reference of each biomarker for HGSOC diagnosis. The error bars denote median±95% CI (confidence interval).

FIG. 13: Response of OCC-DNA nanosensors to protein HGSOC biomarkers, creatinine, and bilirubin in 20% fetal bovine serum. The fluorescence spectra were obtained 2 hours after the incubation. Vertical dashed lines indicate the clinical reference of each serum biomarker for HGSOC screening.

FIGS. 14A and 14B: Relative feature importance of each spectroscopic variable in the HGSOC binary classification models. a, Feature importance of each spectral parameter, used to train the SVM models, of all OCC-DNA sensors in the arrays tested in this work. Solid lines indicate the median feature importance. b, Correlation of averaged F-score with the averaged feature importance of each spectroscopic variable. Vertical dashed lines indicate F-score when all four spectroscopic variables (dint, dint*, dwl, and dwl*) of the OCC-DNA were included as feature vectors in the model development.

FIG. 15: Correlation of F-score and r² of the biomarker prediction models with the relative feature importance of each spectroscopic variable. For the binary classification models (top rows), samples were divided into two groups—abnormal vs. normal levels of serum biomarkers—based on the clinical references (CA125: 50 U/mL, HE4: 150 pM, YKL40: 1650 pM) and assessed the prediction accuracy of abnormal levels of each biomarker. Feature importance of the prediction models shows which spectral parameters most impacted the model performance using an ablation study. Biomarker dependent variables that were identified in FIG. S6 are highlighted in bold. Vertical dashed lines indicate F-score when all four spectroscopic variables (dint, dint*, dwl, and dwl*) of the OCC-DNA were included as feature vectors in the model development.

FIG. 16: Absorption spectrum of the ss(GT)₁₅ wrapped 3,4,5-trifluoroaryl OCC functionalized OCC-DNA complex. The quantification of SWCNT concentration was performed after subtracting the background signal (dashed line). The optical density of the (6,5) SWCNT E₁₁ peak was approximated as 5 μg/mL based on a previous report by Zheng et al.

FIG. 17: Long term stability of OCC-DNAs. Optical density of (6,5) E₁₁ bands of the OCC-DNAs, measured 1 week and 6-10 months after synthesis. The OCC-DNAs in 1×PBS were stored at 4 degrees Celsius (C) until measured.

FIG. 18: Schematic of OCC-DNA synthesis. 1: Unfunctionalized (6,5) SWCNTs were enriched using aqueous phase extraction-based separation and stabilized in 1% sodium dodecyl sulfate in water. 2: OCCs were covalently incorporated into the nanotube sidewall via diazonium chemistry and redispersed in 1% sodium deoxycholate (DOC) solution. 3: DOC-wrapped (6,5)-SWCNTs were resuspended by ss(GT)₁₅. 4: The DNA-wrapped SWCNTs were tip-sonicated in 5-10 mg/mL ss(GT)₁₅ in PBS for 1 hour at 6 W and 4 degrees C. 5: The solution was then ultracentrifuged at 100,000 g for 30 min. 6: The top 85% of the supernatant was then dialyzed in PBS to remove free ss(GT)₁₅. The SWCNT pellet in step 5 was further tip-sonicated in 5-10 mg/mL DNA solution (step 4). The sonication-centrifugation steps were repeated up to 5 times to increase the yield.

FIG. 19: A simplified block diagram of a representative server system and client computer system usable to implement certain embodiments of the present disclosure.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and make part of this disclosure.

Reliably screening for certain medical conditions like cancers, especially during the earlier stages of disease progression, is challenging. For example, ovarian cancer, the second most common gynecologic malignancy worldwide, is responsible for over 184,000 deaths each year. If there is no sign that cancer has spread outside of the ovaries, five-year survival rates are over 90%. However, 59% of cases are diagnosed after they have metastasized to distant sites, for which the 5-year survival drops to only 29%. The earlier detection of ovarian cancer and timely measurements of disease progression and recurrence would markedly improve outcomes.

Conventionally, serum biomarker measurements, such as cancer antigen 125 (CA125) combined with transvaginal ultrasonography, have been suggested for use as a screening tool for the detection of ovarian cancer. Recent reports have found that these methods do not result in early-stage detection and confer little survival benefit in part due to the challenge of improving sensitivity while maintaining high specificity. Other complementary serum biomarkers such as human epidermis protein 4 (HE4), chitinase-3-like protein 1 (YKL40), and mesothelin, or panels of biomarkers have been reported to result in higher sensitivity over CA125-based screening. However, the improvement in discriminatory power for ovarian cancer diagnosis is still under debate. Currently, no screening strategy has been shown to reduce mortality, and screening strategies are associated with a high rate of false-positive results and a risk of harm from invasive testing.

Major factors limiting precise diagnosis using serum biomarkers include patient heterogeneity and low specificity resulting from few established molecular markers. Known biomarkers may not represent the complete disease state or may be present in many other diseases. Thus, accurate detection of analytes does not always confer high sensitivity and specificity for a disease. Many serum biomarkers in clinical practice thus only provide incremental value for treatment options, and often do not reduce the screening cost for patients.

Disclosed are embodiments of an approach that overcomes diagnostic challenges through a perception-based strategy. Nature has evolved perception to identify and interpret multidimensional stimuli against target heterogeneity. Perception achieves target identification by using a number of sensory inputs wherein each encodes certain features of the target and analyzing these inputs against a pre-learned target pattern library. For instance, the perception of smell uses an array of non-specific olfactory receptors, whose pattern of responses is processed by the neural network in our brain to identify an odor. Olfactory receptors are relatively small in number (100-200), yet through perception, they enable recognition of many different odors, far exceeding what is possible with one-to-one recognition. For these odors, although each signal produces relatively little predictive value, the full array of responses processed as a whole nevertheless lead to accurate identification.

Perception-based approaches have been used to classify various disease conditions based on different patterns in methylation of DNA sequencing, volatile organic compounds using electronic noses, small metabolites using mass spectrometry, and image analysis of pathology, computerized tomography scan, and magnetic resonance imaging. Machine learning processes recognize disease-specific patterns that are too subtle or complex to be detected by human eyes or conventional analytical methods and aid in the construction of robust diagnostic models. Despite efforts to develop a generalizable platform of perception-based diagnostic screening using pathology or radioimaging data, challenges remain in the identification of effective disease markers to achieve high sensitivity and selectivity and practical feasibility in the clinic.

Semiconducting single-walled carbon nanotubes (SWCNTs) exhibit intrinsic near-infrared fluorescence with environmental responsivity down to the single-molecule level. The emission of SWCNTs (E₁₁) is sensitive to dielectric environment, redox perturbations, and electrostatic charge. Non-covalent encapsulation with polymers, including short oligonucleotides, facilitates aqueous suspension and confers molecular selectivity to their optical responses via 1) contributing to a molecular masking effect that defines the shape and size of the exposed surface of SWCNTs and 2) modulating their optical bandgaps.

Organic color centers (OCCs) are molecularly tunable quantum defects on SWCNTs which are produced by covalent functionalization of a SWCNT. OCCs efficiently harvest mobile excitons through the SWCNT antenna, producing distinct fluorescence bands (E₁₁-) at longer wavelengths from the E₁₁ band. The E₁₁-fluorescence introduces new biochemical sensitivities to SWCNTs determined by the chemical nature of the defect, making OCCs the molecular focal points for local environmental responses.

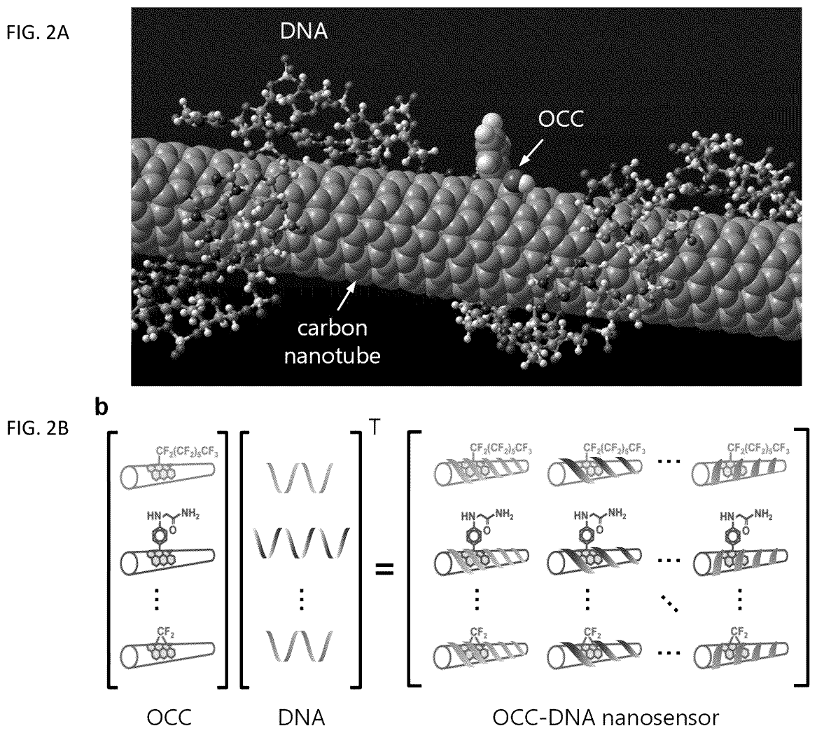

Presented herein are embodiments of a nanosensor array and a computational model that result in the perception-based detection of ovarian cancer (or other medical conditions) from patient serum samples. To transduce broad types of physicochemical properties of a biofluid, the nanosensor arrays may be designed using OCC-functionalized, ssDNA encapsulated SWCNTs (OCC-DNAs, FIGS. 2a and 2b). The emission of the OCC-DNA nanosensors exhibited diverse responses to serum samples collected from patients with high-grade serous ovarian carcinoma (HGSOC), other non-HGSOC diseases (including patients in remission, other gynecologic processes such as endometriosis and low-grade ovarian carcinoma, non-gynecologic cancers, and other conditions), and healthy individuals, but the optical responses did not provide substantial predictive value to differentiate these patients using conventional statistical analyses. The disclosed approached thus trained several machine learning models to classify the three categories of patients using the OCC-DNA sensor array responses. In various embodiments, support vector machine models resulted in striking sensitivity and specificity of HGSOC detection with an accuracy approaching 95%-significantly better than conventional serum biomarker-based identification. Potential interferents, such as drug treatments, were accounted for. The sensors were then used to assess the degree of predictive value conferred by known ovarian cancer serum biomarkers, including CA125, HE4, and YKL40. Support vector regression models showed that the sensor elements responded quantitatively to these markers, but they did not account for all of the predictive value, suggesting that unknown biomarkers play an important role in the differentiation of HGSOC by the sensors.

Although serum biomarkers may be used as diagnostic indicators, they are not specific and/or sensitive enough for screening purposes. Ovarian cancer, for example, is challenging to diagnose, in part because the biomarker levels are elevated in other conditions. In the disclosed approach, a “disease fingerprint” was acquired from patient serum by collecting large data sets of physicochemical interactions to a sensor array composed of organic color center-modified carbon nanotubes. Array responses from 269 patients were used to train and validate machine learning models to differentiate ovarian cancer from other diseases and healthy individuals. This strategy yielded 87% sensitivity at 98% specificity versus 84% via the multimodal test using the biomarker cancer antigen 125 and transvaginal ultrasonography. Detection could not be recapitulated by known protein biomarkers, suggesting that heretofore unidentified biomarkers in the serum milieu are responsible for the sensor response.

The illustrative study discussed herein was directed to ovarian cancer as an example, but the disclosed approach is not so limited, and various embodiments are applicable to other diseases and conditions.

### Overview of Systems and Methods

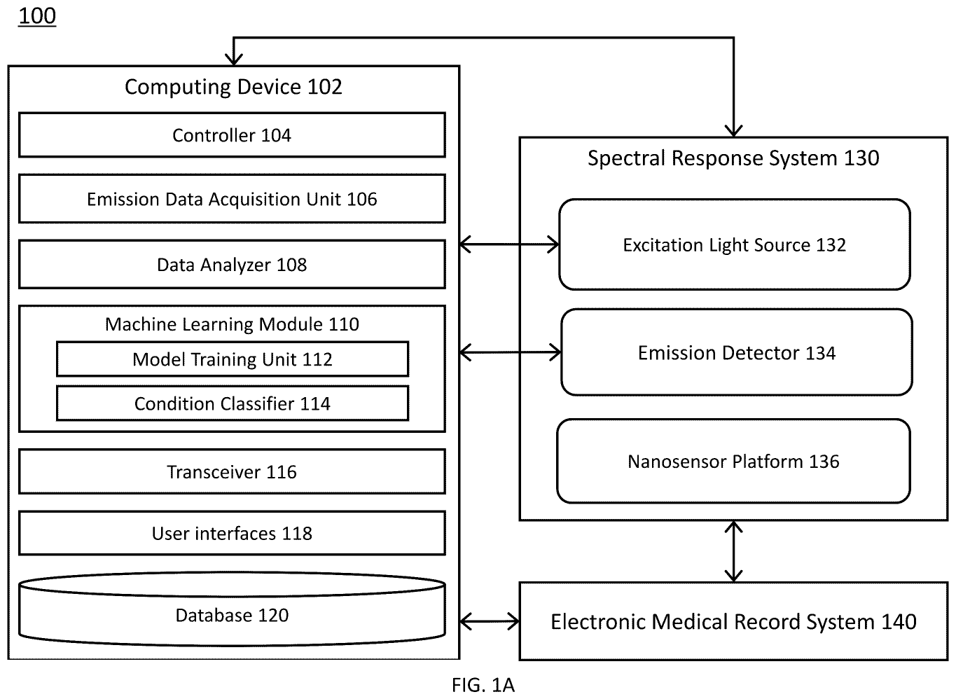

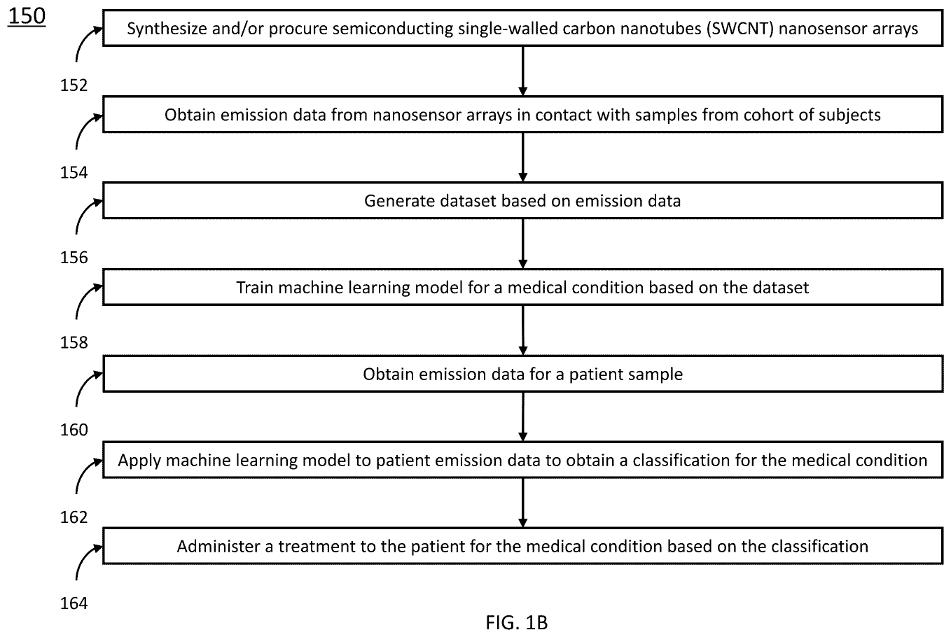

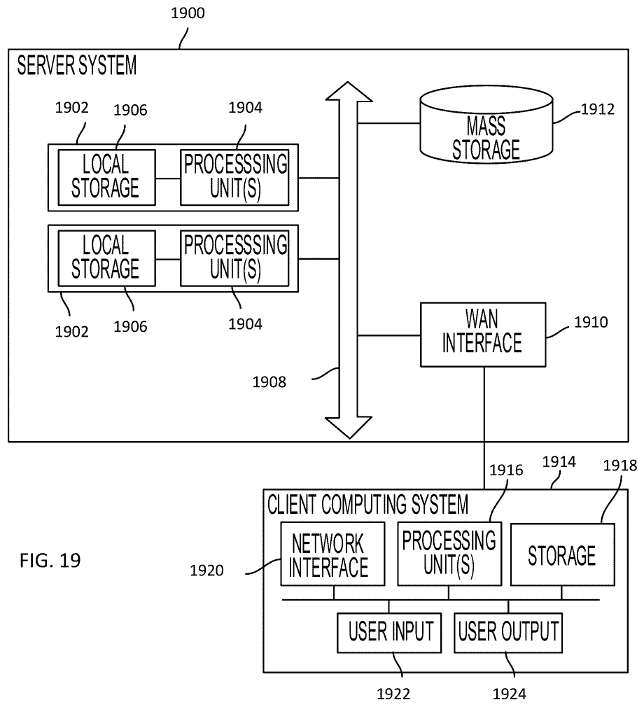

Referring to FIG. 1A, in various embodiments, a system 100 may be used to implement computational models and overall approach disclosed herein. The system 100 may include a computing device 102 (or multiple computing devices, co-located or remote to each other) and a spectral response system 130. The spectral response system 130 may include, for example, one or more excitation light sources 132 (e.g., coherent light emitters such as a laser with a frequency such as 575 nanometers (nm)) and one or more emission detectors 134 (comprising sensors capable of detecting light at various frequencies, such as infrared fluorescence from excited molecules in a sample. The spectral response system may include a nanosensor platform 136, which may include vessels that include nanosensors and that are capable of receiving biological samples (e.g., biofluids from study subjects or patients) that are to be brought into contact with the nanosensors. Molecules in the samples in the nanosensor platform 136 may receive excitation light from the excitation light source 132, and the emission detector may detect emissions from the molecules in the samples. In various implementations, the components of the spectral response system 130 (e.g., excitation light source 132, emission detector 134, and/or nanosensor platform 136) may be separate components in various combinations and arrangements, such that the spectral response system 130 may be one system, two separate systems, or three separate systems. In certain implementations, computing device 102 (or components thereof) may be integrated with one or more of the spectral response systems 130 (or components thereof). In various potential setups, with reference to FIG. 19, one or more of the computing devices 102 may correspond to server system 1900 that receives emission data from a client computing system 1914 (which may be, or may comprise, a spectral response system 130 and/or components thereof), and/or to a client computing system 1914 that sends emission data and analyses to a server system 1900.

The computing device 102 (or multiple computing devices) may be used to control and/or receive signals acquired via spectral response system 130 (and/or components thereof directly). In certain implementations, computing system 102 may be used to control and/or receive signals acquired via spectral response system 130. The computing device 102 may include one or more processors and one or more volatile and non-volatile memories for storing computing code and data that are captured, acquired, recorded, and/or generated. The computing device 102 may include a controller 104 that is configured to exchange control signals with spectral response system 130 and/or components thereof, allowing the computing device 102 to be used to control, for example, the emission and detection of light at the spectral response system 130. The computing device 102 may also include an emission data acquisition unit 106 configured to perform, for example, generate and obtain emission data from samples at the nanosensor platform 136. A data analyzer 108 may be configured to perform data analysis functions, such as preprocessing of data and data analysis for determining, for example, changes in intensity, wavelength, or other parameters. A machine learning module 110 may be used to implement the generation and application of models and classifiers as disclosed therein. A model training unit 1112 may be used to generate training datasets from raw emission data (e.g., from emission data acquisition unit 106) and/or processed emission data (e.g., from data analyzer 108) and train models using various machine learning techniques. A condition classifier 114 may be configured to apply the trained models from model training unit 112 to patient data to determine a classification for the medical condition in patients, such as the presence or absence of a cancer or other medical condition.

A transceiver 116 allows the computing device 102 to exchange readings, control commands, and/or other data with spectral response system 130 and/or components thereof, wirelessly and/or via wires. One or more user interfaces 118 allow the computing system 102 (or components thereof) to receive user inputs (e.g., via a keyboard, touchscreen, microphone, camera, etc.) and provide outputs (e.g., via a display screen, audio speakers, etc.). The computing device 102 may additionally include one or more databases 120 for storing, for example, signals acquired via one or more sensors, raw and processed data, and results of analyses. In some implementations, database 120 (or portions thereof) may alternatively or additionally be part of another computing device that is co-located or remote and in communication with computing device 102 and/or with spectral response system 130 (and/or components thereof).

In various implementations, the system 100 may include an electronic medical record (EMR) system 140 with clinical data related to study subjects and/or patients. The machine learning module 110 may receive clinical data from EMR system 140 in training computational models for classifying medical conditions. The EMR system 140 may include data on whether study subjects have the medical condition when, for example, training a model using supervised machine learning techniques.

With reference to FIG. 1B, an example medical condition classification process 150 is illustrated, according to various potential embodiments. Process 150, or parts thereof, may be implemented by or via one or more computing devices 102. At 152, if they are not already synthesized or procured, semiconducting single-walled carbon nanotubes (SWCNT) nanosensor arrays may be synthesized and/or procured. The nanosensor arrays and manufacture thereof is further discussed below. Once the nanosensor arrays are available, they may be placed into contact with samples (e.g., biofluids such as sera) in, for example, the nanosensor platform 136. At 154, the computing device 102 and the spectral response system 130 may be used to obtain emission data from the nanosensor array platform 136. This may be accomplished by, for example, having controller 104 send a control signal to the spectral response system 130, or the excitation light source 132, to emit an excitation light at the sample. The fluorescent or other light emanating from the sample in the nanosensor platform 136 can be detected using emission detector 134, and the detected signals or other data based on the light detected using emission detector 134 can be transmitted to the computing device 102.

At 154, the signals or other data based on emissions from the nanosensor platform 136, can be processed and used (e.g., by or via data analyzer 108) in generating a dataset. At 156, the dataset and/or other data (raw or processed) can be used (by or via, e.g., model training unit 112) to train a machine learning model for a particular medical condition. In various embodiments, the model may be trained using data from, for example, EMR system 140. At 158, emission data for a patient sample may be obtained using computing system 102 and spectral response system 130. The patient sample may be from a patient who is being evaluated for the medical condition. The patient sample may be placed in the nanosensor platform 136 as discussed with respect to the subjects in the cohort involved in training of the model, and molecules in the sample can be excited using excitation light source 132 and light from the sample can be detected using emission detector 134.

At 162, the trained machine learning model may applied to the emission data corresponding to the patient sample. This may be accomplished by or via, for example, condition classifier 114, which may receive the trained model from model training unit 112. At 164, a classification obtained by using the trained model on the emission data from the patient sample (e.g., whether the patient has a medical condition and a confidence score in the classification) may be used by a clinician to administer a treatment or otherwise make decisions with respect to the patient and potential treatment protocols with respect to the medical condition.

### Nanosensor Array and Computational Models

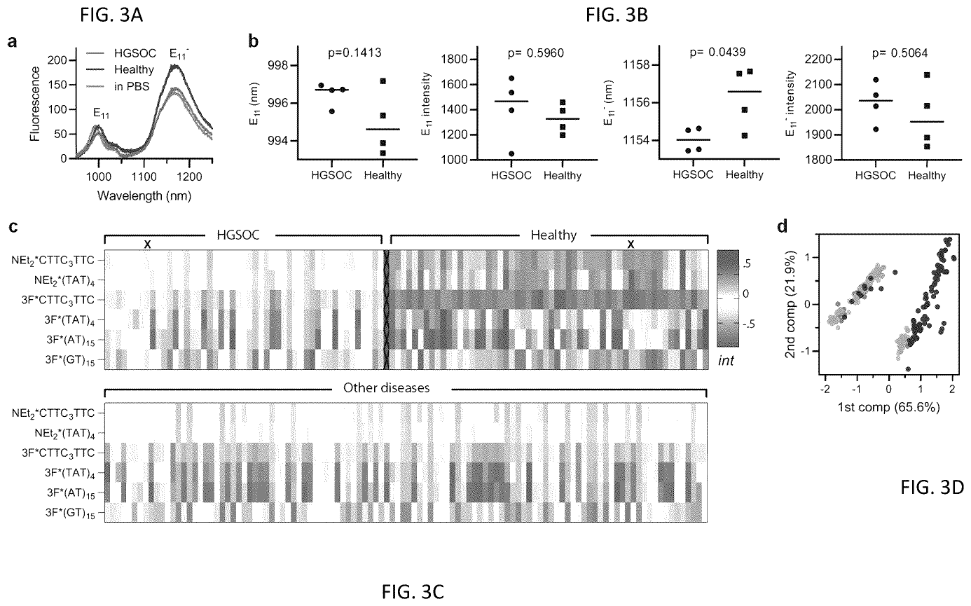

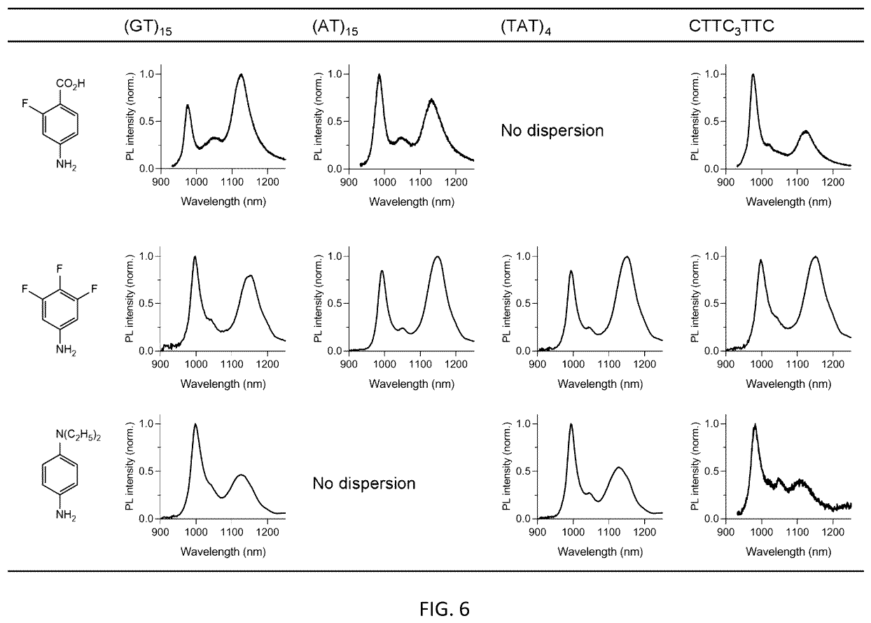

In various embodiments, an array of OCC-DNA nanosensors may be synthesized by introducing several sp³ defects to the (6,5) SWCNT via diazonium chemistry and encapsulating the SWCNT with a library of ssDNA to solubilize the nanosensors in biofluids. The ssDNA sequences may be chosen based on the recognition sequences of DNA that form specific wrapping patterns on the SWCNT surface to result in diverse, highly-defined surface morphologies to confer disparate sensitivities to the local environment. In a study, ten different OCC-DNA nanosensors were successfully synthesized from the combinations of three OCCs and four DNA sequences (Table 1). Each OCC-DNA nanosensor featured a pair of emission peaks depending on the chemical nature of the OCC and DNA sequence. In the study, 575 nm excitation was used to selectively excite (6,5)-SWCNT (FIG. 3a and FIG. 6), resulting in emission at ˜1000 nm from the (6,5) nanotube species E₁₁ band and a peak falling between 1110 to 1170 nm, depending on the aryl functional group. The latter is denoted as the E₁₁-band, or “OCC peak.”

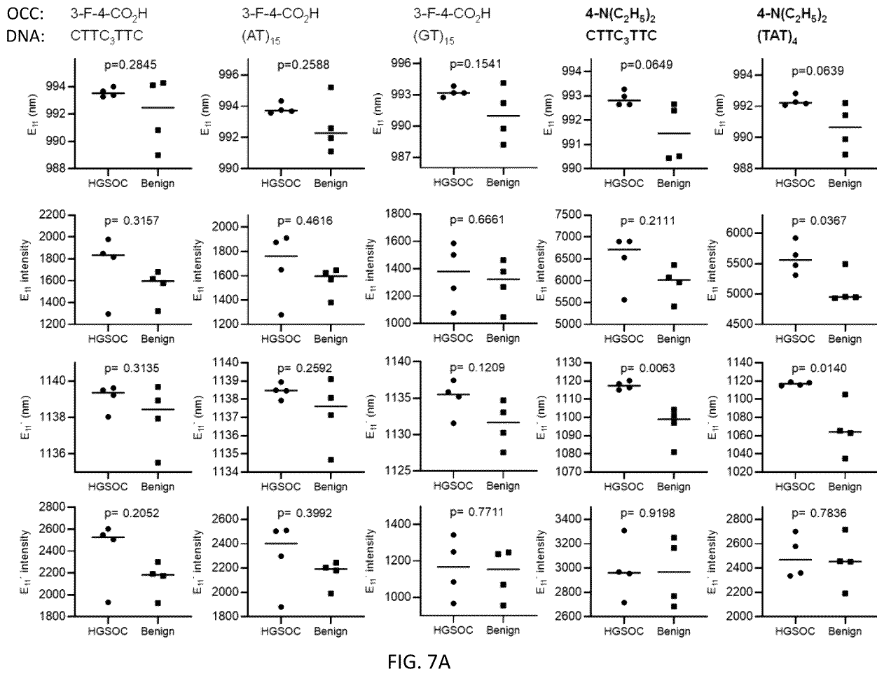

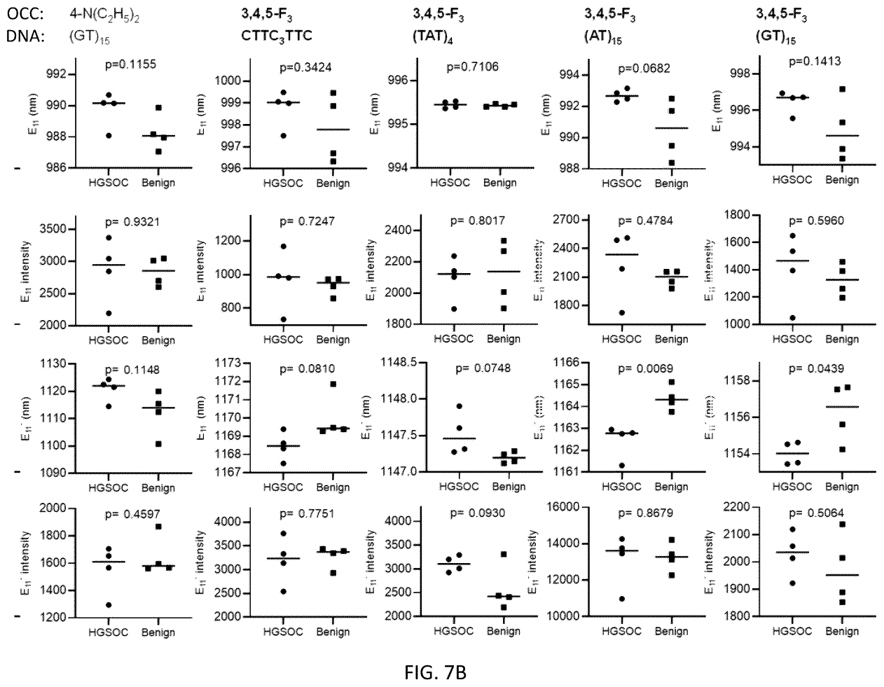

To determine a minimal set of OCC-DNA combinations that provided the most diverse responses from the patient samples, the study measured the fluorescence spectral responses of the OCC-DNAs to serum samples from HGSOC patients and healthy individuals. Four serum samples of the two conditions were incubated with ten different OCC-DNAs for 2 hours, and the fluorescence spectra of the OCC-DNA complexes were acquired. For each OCC-DNA nanosensor, the study analyzed four different spectral features of the OCC-DNA nanosensors that were modulated in response to interactions with analytes in serum: E₁₁ and E₁₁-intensity (int and int*) and wavelength (wl and wl*). From these data, the study identified the sensors that gave statistically significant differences in response to healthy versus cancer groups in parametric t-tests (quantified by p-value, FIG. 3b and FIG. 7). OCC-DNAs which perform well independently may make good choices when used in combination. Six OCC-DNA nanosensors exhibiting E₁₁ or E₁₁-peak wavelengths with statistically significant differences between HGSOC and healthy groups (p-values<0.10) were selected for the sensor array used in the subsequent parts of this study (highlighted in Table 1, FIG. 7). The selection/reduction of features improves the training speed and model performance by eliminating redundant features in the data set.

TABLE 1
OCC-DNA nanosensor elements. Left: Chemical diversity of OCCs
with varying terminating moieties on the aryl functional group.
Center: Special oligonucleotide sequences that form molecular
masks on CNTs. Right: Synthesized OCC-DNA nanosensors. A sensor
array comprised of multiple OCC-DNA nanosensors and was used for
the machine learning training (placed in brackets “{_}”). NEt₂,
3F, and F—CO₂H represents 4-N,N-diethylamino, 3,4,5-trifluoro,
and 3-fluoro-4-carboxy aryl organic color centers, respectively.

| Terminating group | | |
|---|---|---|
| of aryl OCC | ssDNA sequence | OCC-DNA nanosensor |
| -4-N(C₂H₅)₂ | CTTC₃TTC | {NEt₂*CTTC₃TTC} |
| | (TAT)₄ | {NEt₂*(TAT)₄} |
| | (GT)₁₅ | NEt₂*(GT)₁₅ |
| -3,4,5-F₃ | CTTC₃TTC | {3F*CTTC₃TTC} |
| | (TAT)₄ | {3F*(TAT)₄} |
| | (AT)₁₅ | {3F*(AT)₁₅} |
| | (GT)₁₅ | {3F*(GT)₁₅} |
| -3-F-4-CO₂H | CTTC₃TTC | F—CO₂H*CTTC₃TTC |
| | (AT)₁₅ | F—CO₂H *(AT)₁₅ |
| | (GT)₁₅ | F—CO₂H *(GT)₁₅ |

The study initially exposed the OCC-DNA sensor array to 215 patient serum samples and constructed a data set comprised of the spectral feature changes caused by the serum environment. Specifically, the size of the data matrix was Nₛₐ×(N_{f}×N_{OCC-DNA}), where Nₛₐ is the number of serum samples, N_{f} is the number of features per OCC-DNA, and N_{OCC-DNA} is the number of different OCC-DNA complexes in the array. The set of serum samples was collected from 49 HGSOC, 51 other gynecologic diseases (such as endometriosis and low-grade ovarian carcinoma), 29 non-gynecologic cancer, 25 cancer patients in remission, including 7 HGSOC, and 61 healthy donors (Table S1). The fluorescence spectra were collected at three time points during incubation: 2 hours, 24 hours, and 72 hours.

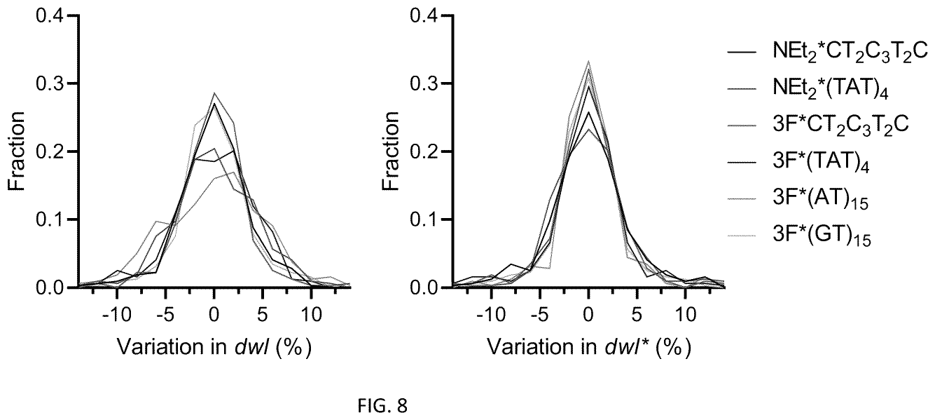

To reduce the inconsistency in spectral measurements, the averaged sensor response of triplicate was used for the data analysis in the study. It is noted that the variation of each measurement from the averaged triplicates was small for all the OCC-DNA peaks (FIG. 8). The variations in dwl and dwl* showed narrow Gaussian distributions with the standard deviations ranging from 3.72-5.37%. The maximum variation in the same sample was less than 15% (<0.3 nm). The analysis confirmed that measurement can reliably identify the small spectral shifts. This is likely because OCC-DNAs exhibit relatively narrow bandwidths (35-80 meV), and thus, small spectral shifts are significantly easier to be resolved as compared to conventional fluorophores (>100 meV).

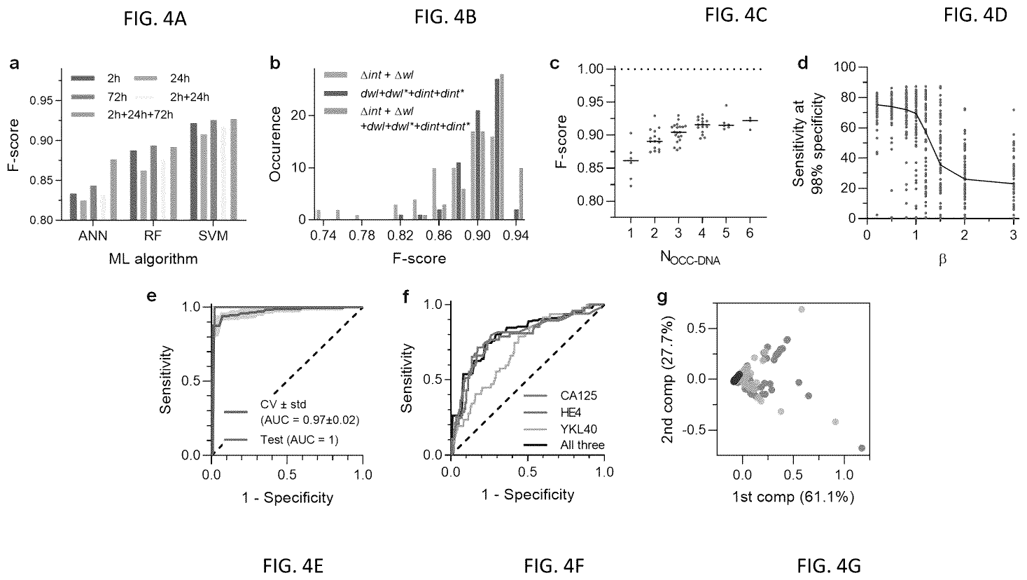

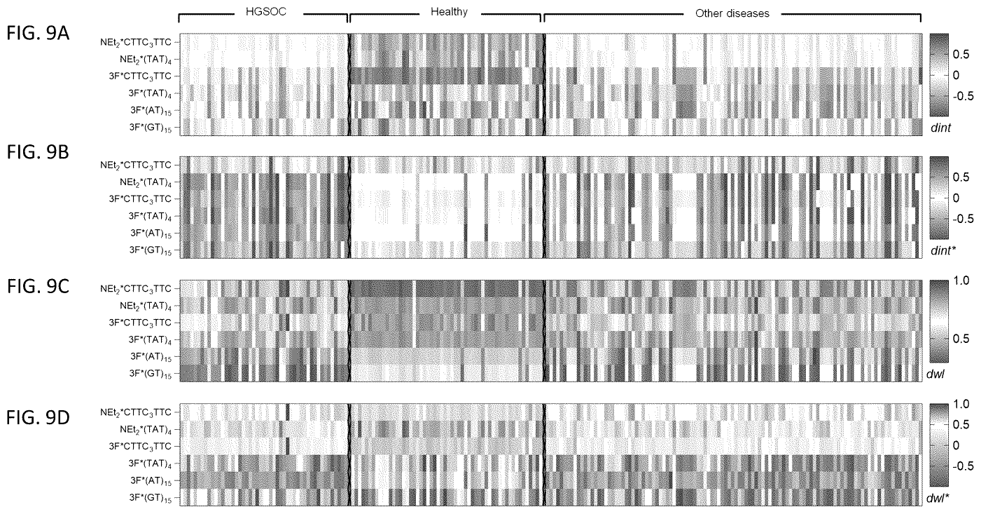

All four spectroscopic variables, int, int*, wl, wl*, measured from the OCC-DNA nanosensor array, exhibited statistically significant differentiation between HGSOC and healthy groups, but the data did not delineate a clear difference between HGSOC and other disease conditions (FIG. 4c, FIG. 9). Principal component analysis (PCA) was performed on the spectroscopic data (N_{f}=4) upon a 2-hour incubation from all combinations of OCC-DNA sensors (N_{OCC-DNA}=6). The first two principal components accounted for 87.5% of the total variance (principal component loadings listed in Table S2). Similar to FIG. 3c, healthy samples showed the differentiable signatures from the disease samples, denoted by their segregation into separate regions in the PCA plot (FIG. 3d), but HGSOC was not separated from other disease conditions.

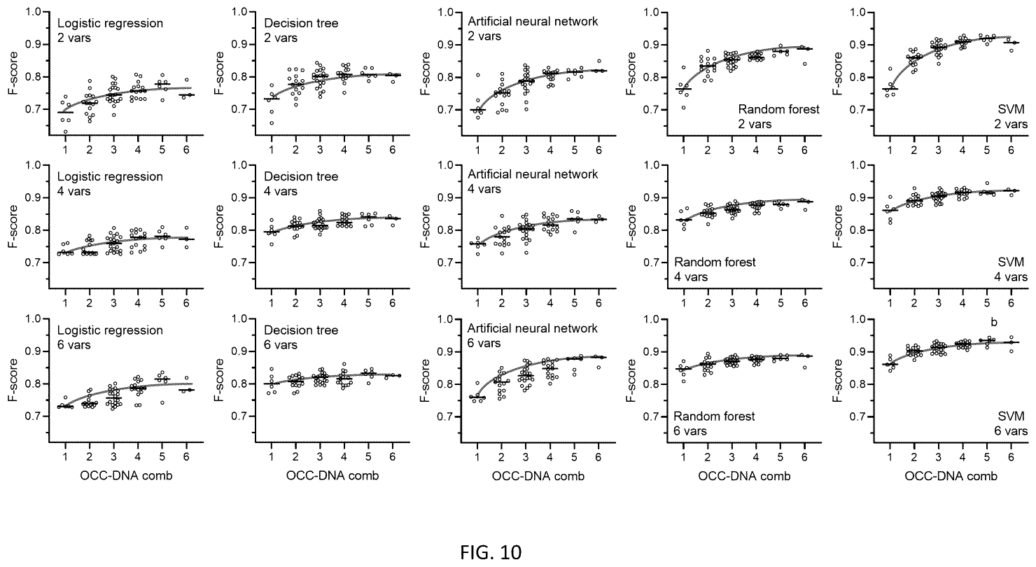

To differentiate HGSOC from other conditions, the study next trained machine learning models using the sensor responses and clinical diagnostic results (FIG. 4, FIG. 10). The algorithms were used for binary classification of sensor responses: HGSOC vs. other diseases+healthy (the differentiation of HGSOC from all other samples). The set of features chosen for the classification task were the spectroscopic variables dint, dint*, dwl, and dwl* collected from the OCC-DNA sensor array. For robustness, the study investigated five standard machine learning algorithms with nested levels of optimization processes: model hyperparameters, model choice, and multilevel validation. The study tested supervised machine learning algorithms: logistic regression, decision tree, artificial neural networks, random forest, and support vector machine (SVM), while tuning models' hyperparameters with Bayesian optimization. The averaged F-score in 10-fold cross-validation was used to assess the model performance (see Methods section below).

The study first examined the machine learning algorithm that most accurately classified HGSOC (FIG. 4a). The study compared the averaged F-scores of the machine learning algorithms using OCC-DNA combinations within the sensor array. The study assessed combinations of OCC-DNA nanosensor responses, up to six at a time, out of the six originally-selected OCC-DNAs (1≤N_{OCC-DNA}≤6), for 63 total possible combinations for each incubation duration (see Table S3). The study found that SVM resulted in the best F-scores among the five machine learning algorithms that were tested (FIG. 10). Thus, the study used SVM models for subsequent optimizations of the HGSOC classifier.

For a second optimization, the study compared the differences in model performance using sensor responses measured under different durations of incubation with the serum samples (N_{f}=3×63). In all the tested machine learning algorithms, there were no statistically significant differences between incubation times (FIG. 10). The study found that combining data sets obtained over multiple incubation durations can improve the model performance, but the performance was only marginally better than using 2 hours of serum incubation (FIG. 4a, Table S3). Thus, the study used the 2-hour data set for subsequent model development for simplicity.

Thirdly, the study examined which spectroscopic variables in the set of feature vectors optimized F-scores. The study compared three combinations of spectral variables, involving the E₁₁-to E₁₁ intensity ratio (Δint), the wavelength difference between E₁₁- and E₁₁ peaks (Δwl), dwl, dwl*, dint, and dint*, and combinations thereof (N_{f}=(2, 4, or 6)×63, FIG. 4b). The SVM models trained with the data set of 2 variables, Δint and Δwl, resulted in lower F-scores. The study found no statistically significant difference between 4 and 6 variables in the F-scores of the optimized SVM models potentially because Δint and Δwl are derivative of the others. Thus, the study used the 4 variables for further investigations.

The study then investigated the impact of the number of different OCC-DNA sensors in the array on the F-score (1≤N_{OCC-DNA}≤6, FIG. 4c). When more OCC-DNA elements were added to the sensor array, the F-scores tended to increase systematically. The trend was the same regardless of which machine learning algorithm was used (FIG. 10). In the study, the best SVM model was trained by the spectral response of five OCC-DNAs: NEt₂*CTTC₃TTC, NEt₂*(TAT)₄, 3F*(TAT)₄, 3F*(AT)₁₅, and 3F*(GT)₁₅. The averaged cross-validation score of the SVM model was 93.9% sensitivity, 95.2% specificity, and an F-score of 0.945 differentiating HGSOC from all other disease+healthy samples. Small variances in F-score and sensitivity (<0.1) in cross-validations suggest that the optimized models are generalizable within the sample set.

Lastly, the study examined if tuning the hyperparameters to maximize the Fp score can improve sensitivity at a high specificity (FIG. 4d). The Fp score is the weighted harmonic mean of PPV and sensitivity and R is chosen such that sensitivity is considered β-times as important as PPV. At decreasing R from 3 to 0.2, sensitivity at 98% specificity systematically increased, although the improvement was statistically insignificant. The best performing prediction model was the sensor array combination of 4-N(C₂H₅)2*CT₂C₃T₂C, 4-N(C₂H₅)₂*(TAT)₄, 3,4,5-F3*(TAT)₄, 3,4,5-F3*(AT)₁₅, and 3,4,5-F3*(GT)₁₅, and yielded 87% sensitivity at 98% specificity when PPV and sensitivity were equally weighted (β=1).

To further assess the robustness of the sensor array and algorithm, the study synthesized a new batch of OCC-DNAs under the same conditions and collected the sensor array response data to an independent test set of 54 patient samples (Nₛₐ=54). To evaluate the model performance in various medical conditions, the test set was sampled from different patients, comprised of 7 HGSOC, 5 other gynecologic diseases, 32 non-gynecologic diseases, and 10 heathy patients. With this new sample set, the optimized SVM model resulted in 100% sensitivity at 98% specificity and an F-score of 0.978. These values are consistent with the cross-validation scores and gave a similar receiver operating characteristic (ROC) curve (FIG. 4e), indicating that the model did not overfit the data.

The risk of bias in the study was evaluated based on Prediction model Risk Of Bias Assessment, PROBLAST42. The risk of bias scored low in terms of predictors, outcomes, and analysis. In participants, the tool resulted in the finding of no systemic differences between training and cross-validation sets. However, the limited medical record of healthy donors and the enriched fraction of breast cancers in the non-HGSOC group of the test set may introduce systematic bias in participant selection and the validation of machine learning models, respectively. For clinical translation of the technology, these risk of bias must be taken into account.

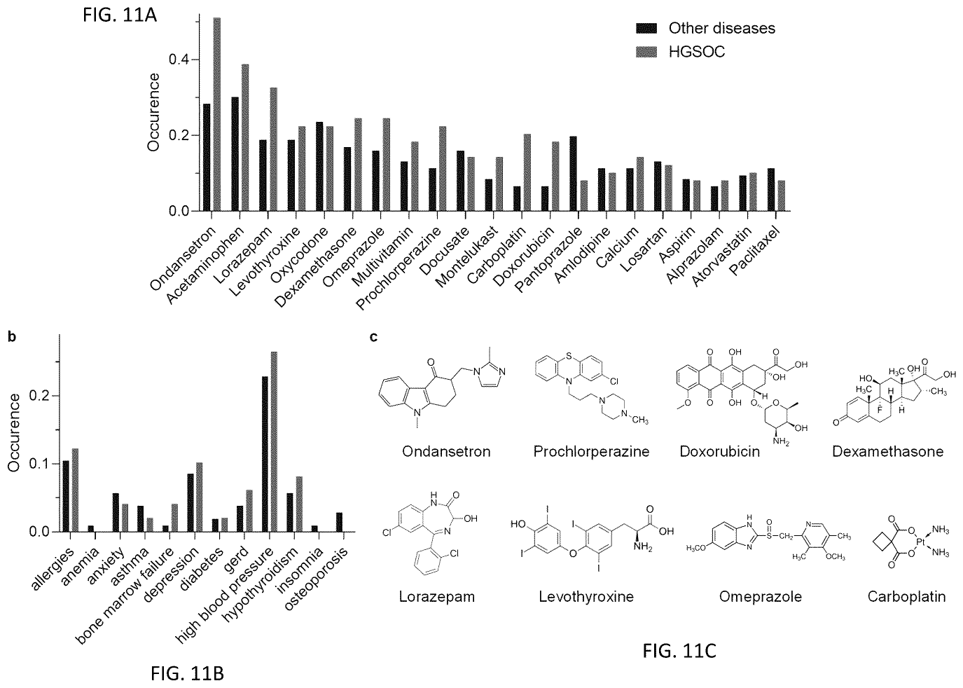

The study also endeavored to account for chemical interferents and background chronic conditions that could confer a bias in the sensor response. From a patient chart review, the study identified chronic diseases and most-common medications administered to the patients (FIG. 11). The study found that 75% of HGSOC and 68% of other disease patients suffered from at least one chronic condition, and the relative abundance between these disease groups was similar. Regarding the medications, the study statistically assessed the contribution of each interferent to the sensor results using a multivariate regression model (Table S5). The regression model determined a linear correlation between the sensor response and medication, using estimated parameters and errors. The adjusted R² of the regression model ranged from −0.045 to 0.233, indicating weak linear correlations of each sensor response to medications. The study confirmed that the sensor array platform accurately classified the disease status regardless of medication and chronic conditions, evidenced by high F-scores of the HGSOC prediction models (Table S3). The analysis suggested no indications that such interferences reduced specificity in HGSOC detection by the sensor platform.

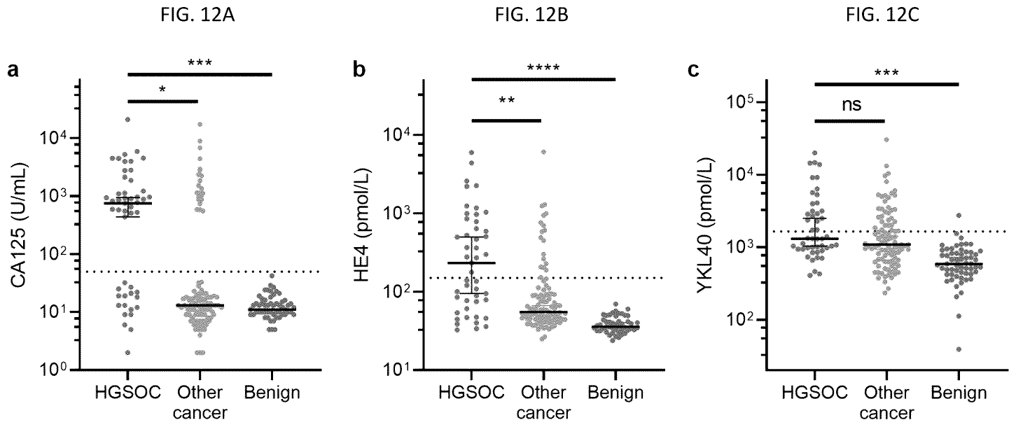

To test the utility of the SVM model relative to conventional diagnostic methods, the study compared conventional biomarker-based HGSOC detection and histology results to the F-score predicted by the SVM model. The study measured known biomarkers in the patient serum samples, including CA125, HE4, and YKL40, creatinine, and bilirubin by immunoassays (see Methods section below). The study assessed the diagnostic accuracy of serum HGSOC biomarkers in these patients (FIG. 4f). Although the differences in serum CA125, HE4, and YKL40 levels, with respect to the clinical references, were statistically significant between HGSOC, healthy, and other (non-HGSOC) diseases (FIG. 12), false-positive rates were high. For example, CA125-based screening with 50 U/mL cutoff resulted in 65.3% sensitivity, 88.3% specificity, and an F-score of 0.621 in the patient sample set. The logistic regression of additional biomarkers marginally improved the HGSOC prediction (FIG. 4f). PCA plots of HGSOC biomarkers CA125, HE4, and YKL40 showed that these markers failed to differentiate HGSOC from other diseases while the healthy individuals' samples clustered together (FIG. 4g). Clinical trials using these biomarkers showed similar results. These results confirmed that the disclosed perception/sensor-based platform significantly outperformed established serum biomarker-based classification, and the accuracy was much closer to diagnosis by the physician (using pathology, imaging, etc.).

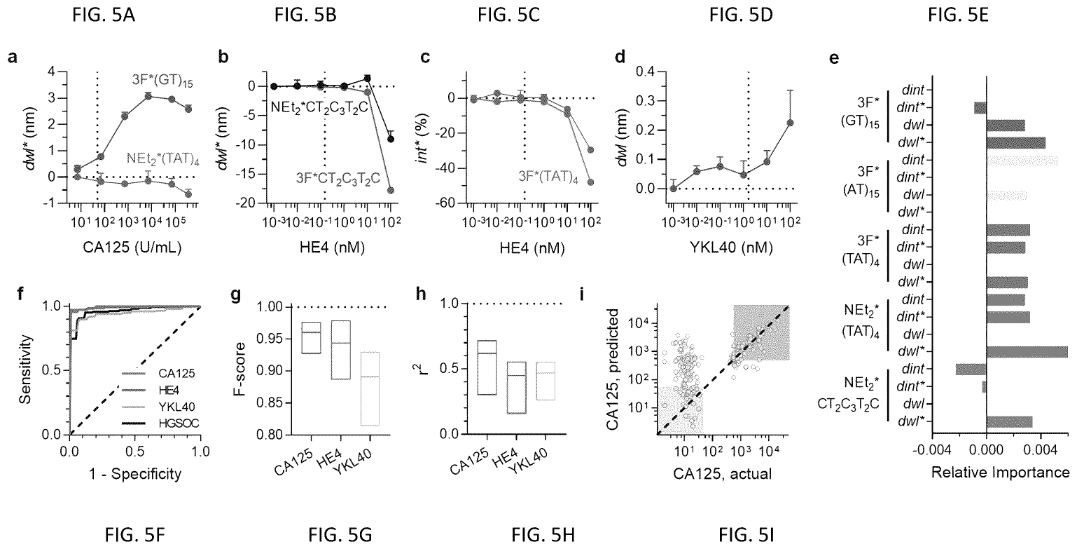

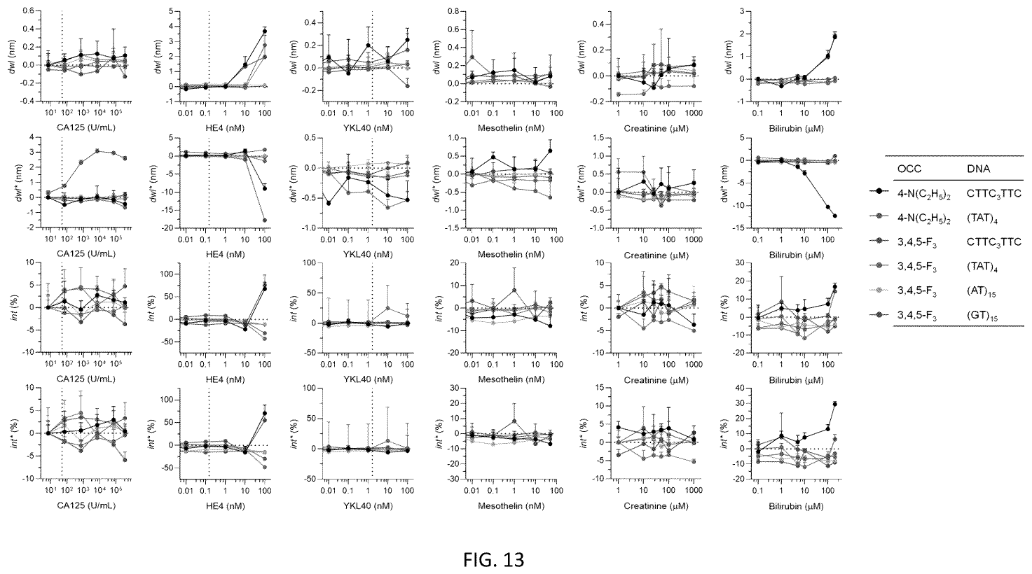

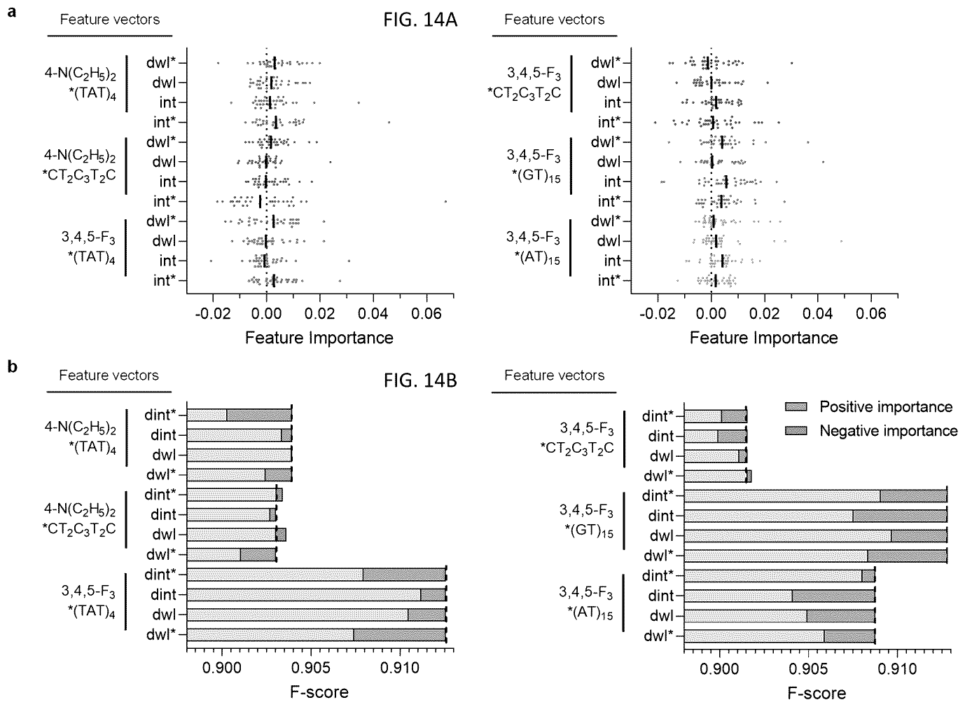

To investigate the molecular basis for the sensor-based HGSOC fingerprint, the study investigated the sensor response to serum biomarkers (FIG. 5). The study measured the spectral response of the OCC-DNA nanosensors upon single analyte titration with bilirubin, creatinine, and HGSOC serum biomarkers, including CA125, HE4, YKL40, and mesothelin in 20% fetal bovine serum (FIG. 5a-d, FIG. 13). The study found that several OCC-DNA spectral responses correlated with CA125, HE4, YKL40, and bilirubin concentrations while mesothelin and creatinine showed no quantitative correlations with the sensor responses. Because of this correlation, the inclusion of biomarker-dependent spectroscopic variables in the training data set likely improved F-scores for HGSOC identification. The study then assessed the relative contribution of each spectral parameter to the model performance by an ablation study-individually dropping each spectroscopic variable from the analysis (FIG. 14). On analysis of feature importance, the study identified that 3F*(GT)₁₅ and 3F*(TAT)₄ were the most important OCC-DNA nanosensors. The study also found that the same feature in different sensor arrays can improve or reduce the prediction scores (FIG. 14). For instance, the E₁₁-intensity (dint*) of NEt₂*CTT had the highest positive feature importance (improved F-score by 0.067) in the sensor array of 4-N(C₂H₅)₂*CT₂C₃T₂C while the same feature reduced the F-score by 0.018 in the sensor array combination of 4-N(C₂H₅)₂*CT₂C₃T₂C and 3,4,5-F3*(GT)₁₅. Overall, the biomarker-dependent features scored highly, indicating that such features improved the SVM model performance (FIG. 5e). The observations confirmed that 1) OCC-DNA fluorescence transduces broad types of subtle differences in physicochemical properties of physisorbed molecules and 2) known serum biomarkers make up part of the disease fingerprint. However, the use of biomarker-dependent features exclusively did not result in optimal F-scores. The inclusion of certain features that showed no quantitative correlation with known biomarkers improved the model performance. These experiments support the proposition that the OCC-DNA nanosensor array results may have been due, at least in part, to the transduction of heretofore unidentified biomarkers.

To further investigate the correlation between serum biomarker levels and the response of the nanosensor array, the study assessed whether the sensor array responses could be used to train an SVM model to identify abnormal levels of known biomarkers in the patient samples. First, the study trained an SVM classification model to detect elevated CA125 by dividing the patient sera into groups based on the threshold for suspicion of malignancy; normal (0-50 U/mL) vs. high (>50 U/mL) CA125. The CA125 training resulted in high F-scores (>0.92) for all possible sensor array combinations (FIG. 5f, 5g, Table S4). The study similarly assessed HE4 and YKL40 with respect to their clinical references of 150 pM for HE4 and 1650 pM for YKL40, and the study developed binary classification models to differentiate abnormal levels using the SVM algorithm. Both HE4 and YKL40 classification resulted in high F-scores (0.89-0.98 and 0.81-0.93, respectively) for the detection of abnormal biomarker levels.

The study additionally investigated whether support vector regression (SVR) models can quantitatively predict serum biomarker levels using the sensor array (FIG. 5h). The best CA125 regression model, using three OCC-DNAs, NEt₂*(TAT)₄, 3F*(TAT)₄, and 3F*(AT)₁₅ resulted in an average R-squared (r²) value of 0.719 (FIG. 5i). It is noted that the prediction error in the normal concentration range (<50 U/mL) was larger than in the high concentration range. This can be attributed to the fact that the detection limit in the single titration experiment was close to the clinical reference of CA125. The SVR models of HE4 and YKL40 were also constructed, resulting in r₂ values of 0.55 and 0.56, respectively. The SVR models suggest that the known biomarkers influence the sensor responses, but the models were not sensitive enough to reliably predict the exact biomarker levels.

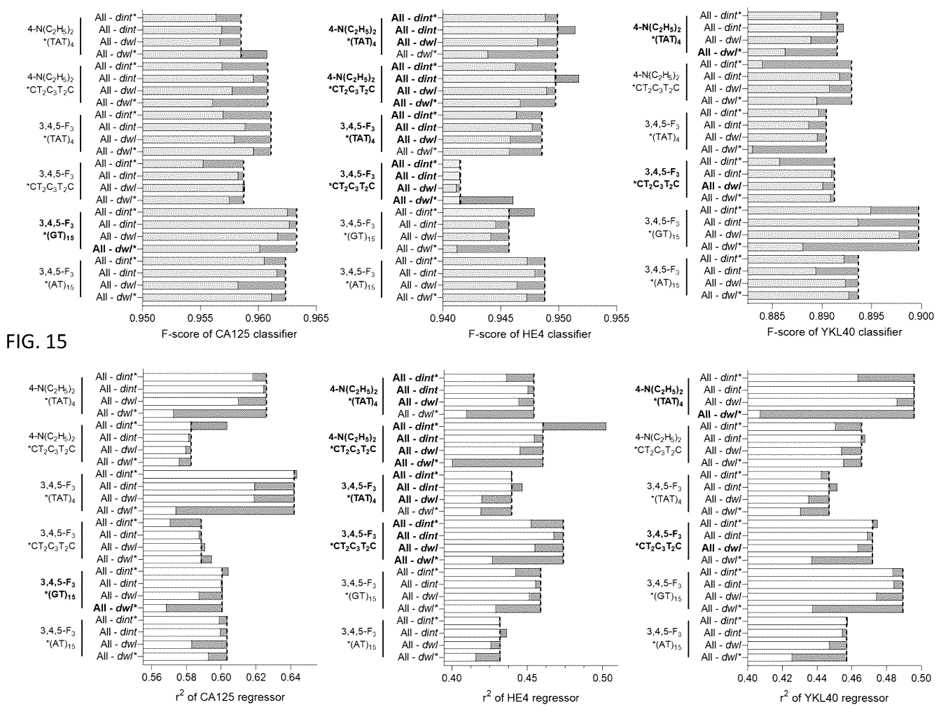

The study assessed the contribution of each spectral parameter to the biomarker classification and regression models (FIG. 15). Most of the spectral parameters had positive relative importance on average, indicating that including such features improved the positive predictive value and sensitivity of the biomarker identification. A positive correlation of the feature importance to F-score (for binary classification) and r² (for regression) was stronger for the biomarker-dependent variables that were identified in the single-analyte experiments (FIG. 5a-d). Regarding bilirubin, however, although OCC-DNA fluorescence responses quantitatively correlated to its concentration over biologically relevant ranges (FIG. 13), small variance of the biomarker levels within the patient samples hindered optimizing a good SVR model for its detection. The SVR model performance for serum creatinine was poor due to a lack of quantitative correlation between sensor response and creatinine concentrations in the single-titrant experiment (FIG. 13).

As disclose above, the study constructed a nanosensor array platform, comprised of OCC-DNA elements and coupled with machine learning algorithms, to investigate the potential to identify HGSOC in patient sera. The array was comprised of multiple OCC moieties and DNA sequences, which together offer a rich design space for modulating the morphology and chemistry of the exposed nanotube surface. The DNA sequence selection was based on the recognition sequences that form specific wrapping patterns on the nanotube surface. These sequences were originally selected to isolate individual (n,m) species/chiralities of nanotubes. It was reasoned that the recognition sequences of DNAs would confer the greatest diversity of interactions with the serum milieu, which is important to establish an OCC-DNA library for screening disease-specific sensor responses. This rationale was based on the findings that ssDNA encapsulates CNTs via π-π stacking interactions, and certain DNA sequences can behave like a “molecular mask” that defines the shape and size of the exposed surface. Their characteristic surface structures are responsible for diverse physicochemical properties of the OCC-DNAs, leading to different protein corona compositions. Different morphologies determined by OCCs and DNA thereby contribute to the selectivity of the nanotube surfaces to the serum milieu. The fluorescence modulation of SWCNTs is caused by several mechanisms including Fermi level shifting through modulation of the immediate redox environment and exciton disruption in response to binding events, which change SWCNT intensity, and solvatochromic (wavelength) shifting due to perturbation of the local dielectric environment, including shifts due to modulation of the local electrostatic environment. OCC fluorescence, on the other hand, is molecularly specific and extremely sensitive to the local chemical environment of the atomic defect sites. Interactions between HGSOC serum biomarkers and OCC-DNA hybrids elicited additional, diverse spectral responses of the sensor array that enabled sufficient differentiation of signals from other sera.

The sensor platform of the study was used to identify HGSOC with high positive and negative predictive values. Model performance of the sensor technology exceeded the results of the current best clinical screening test using longitudinal CA125 and second-line transvaginal ultrasonography (87% vs. 84% clinical sensitivities at 98% specificity). However, because specimens obtained from symptomatic individuals at diagnosis were used in the study for the development and assessment of the technology, prediction outcomes may differ in clinical screening settings in which specimens are obtained in asymptomatic individuals before clinical diagnosis. Evaluation of high-risk cohorts, such as BRCA mutation carriers undergoing risk-reducing surgery, may be used to demonstrate the ability of the technology to identify pre-invasive and early-invasive disease.

The disclosed sensor technology platform exhibits several unique potential advantages for clinical applications. First, this method could be rapidly adapted to the detection of many diseases/conditions. The array could be used to train an algorithm to recognize nearly any disease when given enough data from the sensor responses to the appropriate patient serum samples. Second, this technology could supplement or replace the use of known biomarkers when there are issues with selectivity in conventional multi-analyte tests. Due to the potential to iteratively modify the sensor array and machine learning algorithms and to additively augment training set size, the selectivity may be increasingly optimized. Third, this sensor platform can be used in a high-throughput fashion to facilitate the screening of large populations. Fourth, because the technology does not rely on antibody-based molecular recognition elements, the sensors could be more robust than existing methods, enabling use in resource-limited settings and in technologies such as point-of-care and wearable/implantable devices. Lastly, the sensor technology also may serve as an inexpensive and rapid screening tool to result in a single, easy-to-interpret test result in primary care settings. The materials needed for the sensor cost approximately $5 per sample because of the small amount of OCC-DNAs needed for screening (<5 nanograms). The cost of the sensor measurement would also diminish if measured via high-throughput instruments, and the potential for the use of very-low sample volumes is substantial.

The disclosed approach can employ machine perception to detect disease fingerprints using an array of optical nanosensors. The study carefully investigated the attributes and molecular mechanism that resulted in the striking accuracy of the machine learning-aided nanosensor array. The best-performing HGSOC prediction model (FIG. 4d) in the study included the spectroscopic variables that were not sensitive to the known biomarkers and their relative importance was much significant than the biomarker-related variables (FIG. 14). This suggests that there exist potential biomarkers or combinations thereof that are either unknown or not part of conventional screening approaches but were captured by the OCC-DNA sensor array. Information detailing which biomarkers and molecular interactions primarily result in the disease fingerprint is unknown and largely cannot be determined by current machine learning methods. Quantitative proteomics aided by embodiments of the disclosed nanosensor array may be useful as a discovery tool. Such investigation could potentially be used to facilitate biomarker discovery efforts and uncover new information related to disease pathophysiology.

### Methods in Study

Large scale synthesis of OCC-DNAs: Raw SWCNT material, CoMoCAT SG65 and SG65i (Sigma-Aldrich) was used for the large-scale preparation of OCC-SWCNTs. The SWCNTs were dissolved in chlorosulfonic acid (Sigma-Aldrich, 99.9%) at a concentration of ˜4 mg/mL with magnetic stirring, followed by the addition of an aniline derivative at different molar ratios relative to the SWCNT carbon, and equimolar amounts of sodium nitrite (Sigma Aldrich, ≥97.0%). The aniline derivatives tested for these experiments include 4-amino-2-fluorobenzoic acid (Sigma-Aldrich, 97%), 3,4,5-trifluoroaniline (Sigma-Aldrich, 98%), and N,N-diethyl-p-phenylenediamine (Sigma-Aldrich, 97%). The SWCNT-superacid mixture was then added drop-by-drop into Nanopure water with vigorous stirring (Safety Note: the neutralization process is aggressive; a significant amount of heat and acidic smog can be generated. Personal protective equipment, including goggles/facial mask, lab coats, and acid-resistant gloves, are necessary. The neutralization must be performed in a fume hood). The resulting OCC-SWCNTs instantly precipitate out from the solution. The precipitates were then filtered on an anodic aluminum oxide filtration membrane with a pore size of 0.02 m (Whatman® Anodisc inorganic filter membrane), thoroughly rinsed with Nanopure water, and then dried in a vacuum oven.

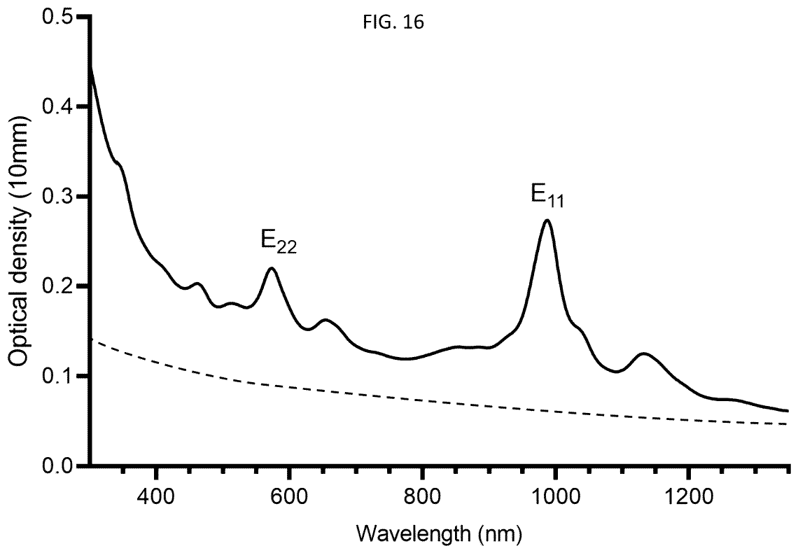

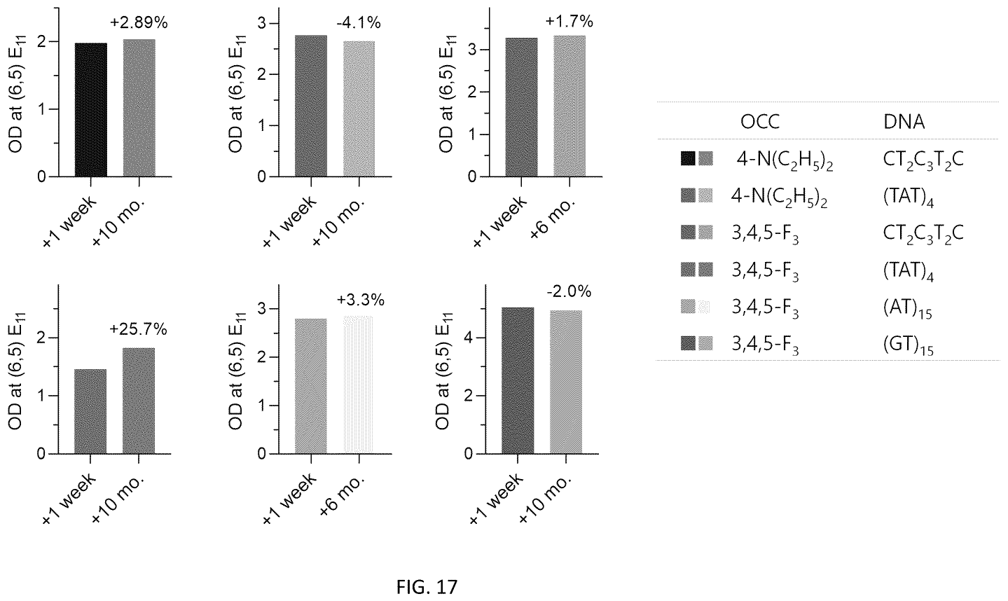

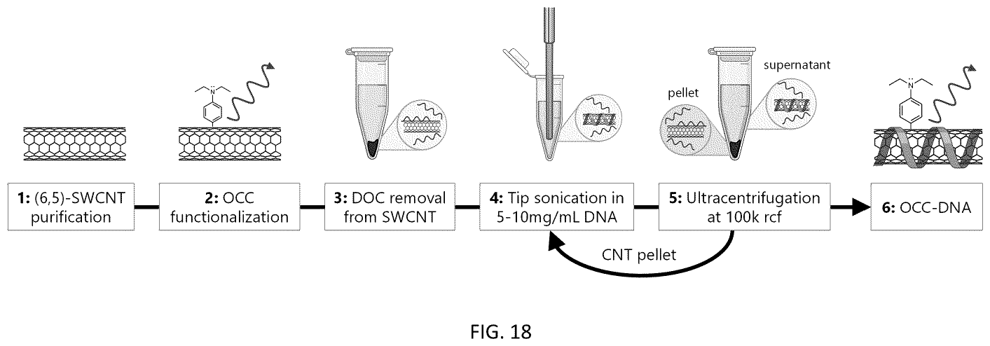

The OCC-SWCNTs were stabilized by 3.5 mg/mL ssDNA in phosphate buffered saline (PBS). The OCC-SWCNT were individually dispersed by ultrasonication at 6 W for 60 min using a probe-tip sonicator (Sonics & Materials, Inc) at 4 degrees C. for 1 hour. The DNA to SWCNT mass ratio is 5 to 1. Then the OCC-DNA solutions were centrifuged at 100,000 g and 4 degrees C. for 30 min. The 80% supernatant was dialyzed against PBS for 36 hours to remove free DNA (Spectra-Por, Float-A-Lyzer, MWCO=1 MDa). The absorption spectra of the dialyzed solutions were collected with a UV-Vis-NIR spectrophotometer (Jasco, Tokyo, Japan). After subtracting background, the optical density at (6,5) E₁₁ (˜1000 nm) was used to estimate the relative OCC-DNA concentration (FIG. 16). The OCC-DNAs were kept at 4 degrees C. until used (up to 6 months) as the OCC-DNAs remained colloidally stable (FIG. 17).

OCC-DNA and serum recombinant protein handling: For the training set data collection, the study used the OCC-DNAs that were synthesized within 6 months prior to testing with patient serum samples (1 week to 6 months old). For the test set, the study used freshly prepared OCC-DNAs (less than 2 weeks old). The OCC-DNA concentration was adjusted to 0.325 mg/L in PBS. The study introduced 20 μL of a patient serum sample to 80 μL of OCC-DNAs in a 96-well plate (Corning) to make the OCC-DNA concentration of 0.26 mg/L in each well. OCC-DNAs in 100 μL PBS (0.26 mg/L) was also prepared to compare the relative changes in sensor response in serum for feature vector construction (See Data preprocessing in Methods). The OCC-DNA was incubated at room temperature for 2 hours and in a cold room (4 degrees C.) after the spectral acquisition at 2-hour time point. Data were taken at three time points during incubation: 2 hours, 24 hours, and 72 hours.

To test sensor sensitivity to serum biomarkers, OCC-DNA complexes were added to a 96-well plate at a concentration of 0.26 mg/L in a 100-1 total volume of 20% FBS (Gibco). In triplicate, the following were added into wells at biologically relevant concentrations: 0-352000 U/mL recombinant human CA125/MUC16 (R&D Systems), 0-100 nM recombinant human HE4 (RayBiotech), 0-100 nM recombinant human YKL40 (R&D Systems), 0-50 nM recombinant human mesothelin (BioLegend), 0-1000 μM creatinine (Fisher Scientific, ≥98%, anhydrous) or 0-200 μM bilirubin (Fisher Scientific, ≥97%). Experiments were performed with the same time points as above. All experiments were performed in triplicate.

High-throughput near-infrared spectroscopy: Fluorescence emission spectra of OCC-DNAs were acquired using a home-built near-infrared fluorescence spectroscopy apparatus consisting of a tunable white light laser source, inverted microscope, and InGaAs NIR detector. The SuperK EXTREME supercontinuum white-light laser source (NKT Photonics) was used with a VARIA variable bandpass filter accessory, capable of tuning the output 500-825 nm, set to a bandwidth of 20 nm centered at 575 nm. The light path was shaped and fed into the back of an inverted IX-71 microscope (Olympus), where it passed through a 20×NIR objective (Olympus) and illuminated the samples in a 96-well plate. Emission from the OCC-DNAs was collected through the 20× objective and passed through a dichroic mirror (875 nm cutoff, Semrock). The light was f/#matched to the spectrometer using several lenses and injected into a Shamrock 303i spectrograph (Andor, Oxford Instruments) with a slit width of 100 μm, which dispersed the emission using a 86 g/mm grating with 1.35 μm blaze wavelength. The spectral range was 723-1694 nm with a resolution of 1.89 nm. The light was collected by an iDus 1.7 μm InGaAs (Andor, Oxford Instruments) with an exposure time of 10 seconds. An HL-3-CAL-EXT halogen calibration light source (Ocean Optics) was used to correct for wavelength-dependent features in the emission intensity arising from the spectrometer, detector, and other optics. A Hg/Ne pencil-style calibration lamp (Newport) was used to calibrate the spectrometer wavelength. Background subtraction was conducted using a well in a 96-well plate filled with PBS or 20% FBS, depending on the experiment. Following acquisition, the data were processed with custom code written in Matlab that applied the aforementioned spectral corrections and background subtraction and was used to fit the data with Lorentzian functions.

Serum sample set: 269 waste samples were collected from female patients diagnosed with ovarian and other cancers under a Review Board approved protocol. From this sample set, 56 specimens were collected from patients diagnosed with high-grade serous ovarian cancer, 71 specimens from healthy donors, 56 with other gynecologic diseases, 61 with non-gynecologic diseases, and 25 in remission. There was no statistically significant difference in age distribution for each group. Diagnoses were identified from a chart review of each patient; all diagnoses included histology and were confirmed by gynecologic oncology attending physician. Patient demographics, diagnosis, and biomarker levels are available in Table S1.

Serum assays: Serum concentrations of CA125 and HE4 were determined on the Abbott Architect i2000 analyzer (Abbott Diagnostics, Abbott Park, IL, USA) using a chemiluminescent microparticle immunoassay. YKL40 was analyzed using a singleplex immunoassay on the Protein Simple Ella system. The Abbott C8000 analyzer was used to determine the concentrations of creatinine by quantitating the formation of creatinine picrate in alkaline conditions, and bilirubin was analyzed by the formation of azobilirubin using the diazo reagent under specified conditions.

Data preprocessing: Quantities representing the sensor response to patient serum were acquired by the Lorentzian fitting of OCC-DNA fluorescence spectra: E₁₁ intensity, E₁₁-intensity, E₁₁ wavelength, and E₁₁-wavelength. The average value of triplicates was used as feature data for machine learning processes. Feature values were defined as a difference in sensor response acquired from patient serum and PBS. Specifically, the E₁₁ peak position feature, dwl, was defined as the wavelength difference between the E₁₁ peak in the patient sample, wl, and PBS, wl0, dwl=wl−wl0. The E₁₁ peak intensity feature, dint, was normalized as dint=int/int0, where int and int0 are the E₁₁ peak intensity in serum and PBS, respectively. Similarly, the study defined E₁₁-peak related features, dwl* and dint*, indicating the relative E₁₁-peak position and intensity. The study additionally considered the relative change in E₁₁- to E₁₁ intensity, zint=(int*/int)(int0*/int0)⁻¹−1, and the wavelength difference between two peaks, Δwl=dwl*−dwl to check if the addition of these features would create a larger variance in HGSOC prediction.

The study normalized each feature vector to be in the range of [−1, 1] to balance the feature contribution to the model. The imbalance in the size of each group was corrected by upscaling minority species (SMOTE: Synthetic Minority Oversampling Technique) so that the prediction models were not biased by groups with a larger sample size. For the biomarker prediction models, the study divided the data into normal versus high biomarker level groups based on the clinical references (CA125: 50 U/mL, HE4: 150 pM, YKL40: 1650 pM) and corrected the group size using SMOTE.

Model training and performance assessment: Using algorithms implemented in Scikit-Learn, models were created based on Decision Tree, Logistic Regression, Artificial Neural Networks, Random Forest, and Support Vector Machine (SVM) for binary classification. Hyperparameters for each model were optimized using Bayesian Optimization, implemented in the HyperOpt library. The loss function to minimize in the hyperparameter optimization was set to (1−F-score). F-score (or F₁-score) is a measure of accuracy in binary classification and calculated from the harmonic mean of the positive predictive value (PPV) and sensitivity: 2/(sensitivity⁻¹+PPV⁻¹). To rule out the possible overfitting in machine learning process, model performance was evaluated using ten-fold cross-validation. In the cross-validation process, stratified shuffle split validation was used to randomly partition the data set into ten subsamples. In each partition, nine of the ten subsamples were used to train the model, while a single subsample was used to test the trained model. The average F-score of the ten-fold cross-validation was used to assess model performance. The trained models were then tested with an independent set of patient sera (N=54), sampled from different patients (test set), as external validation. Support vector regression (SVR) was used to construct the regression models of HGSOC serum biomarkers with 10-fold cross-validation. The loss function in the hyperparameter optimization was (1−r²). For SVM and SVR, a radial basis function kernel was used and the hyperparameter optimization was performed for the regularization parameter (cost) and the kernel coefficient (gamma) with the maximum iteration of 1000. The hyperparameter space of each machine learning algorithm for model optimization is shown in Table S6.

### Additional Potential Methods for Preparation of SWCNTs

Purification of (6,5) SWCNT. Raw SWCNT material, CoMoCAT SG65i (Sigma-Aldrich) may be dispersed in 1 wt/v % sodium deoxycholate (DOC, Sigma-Aldrich, 99.9%) aqueous solution at a nanotube concentration of 1 mg/mL using tip-sonication at 6 W (Sonics & Materials, Inc) and 4 degrees C. for 1 hour, followed by ultracentrifugation at 100,000 g for 30 min. The 85% supernatant may be used to obtain (6,5) enriched SWCNT solution based on the previously reported protocol. The final purified (6,5) SWCNTs may be stabilized in 1.04% DOC solution to maintain long-term colloidal stability (>6 months).

Covalent functionalization of purified (6,5) SWCNT. N,N-diethlyanimoaryl OCC may be covalently functionalized to the purified (6,5) enriched SWCNTs via diazonium chemistry. N,N-diethylanimobenzene tetrafluoroborate may be freshly synthesized from N,N-diethyl-p-phenylenediamine (97%, Sigma Aldrich) and nitrous acid following a modified literature method. The purified SWCNT solution may be diluted with 1% sodium dodecyl sulfate (SDS, ≥99.0%, Sigma Aldrich) and mixed with the synthesized diazonium salts at the diazonium salt to carbon of (6,5) SWCNT molar ratio of 3.17 to 1. To improve the yield of the diazonium reaction, the SWCNT and diazonium mixture may be illuminated with a mercury arc lamp (X-Cite 120Q, Excelitas) at room temperature. After 20 minutes of illumination, the diazonium reaction may be quenched by diluting the SWCNT solution with 1.04% DOC solution. The functionalized SWCNT solution may be ultrafiltrated using Amicon® Ultra filters (100 kDa MWKO) to remove unreacted diazonium salts and concentrate the OCC-SWCNT solution for DNA rewrapping.

DNA DOC exchange for the OCC-functionalized SWCNTs. To redisperse the functionalized OCC-SWCNTs to biocompatible polymers, the following approach may be used. First, the approach may sequentially add 25 μL of 25 w/v % polyacrylamide (10 kDa, Sigma-Aldrich), 30 μL of 10 mg/mL ssDNA (sequence=5′-GTGTGTGTGTGTGTGTGTGTGTGTGTGTGT-3′, Integrated DNA Technologies), 270 μL of methanol (Anhydrous, 99.8%, Sigma Aldrich), and 600 μL of 2-propanol (99.9%, Sigma Aldrich) to the OCC-SWCNT solution. To precipitate DNA/polyacrylamide encapsulated OCC-SWCNTs, the solution may be centrifuged at 17,000 g for 2 seconds. The supernatant may be further centrifuged for 2 minutes at the same speed and room temperature. The pellets from each centrifugation may be combined and redispersed with 150 μL of water. The addition of 600 μL of 2-propanol and centrifugation may be repeated one more time to remove >98% of DOC.

To improve the stability of DNA wrapping, the OCC-SWCNT pellets may then be diluted in 1 mL of 4 mg/mL DNA in PBS buffer and tip-sonicated for 1-hour at 6 W and 4 degrees C. The OCC-DNA solutions may then be ultracentrifuged at 100,000 g and 4 degrees C. for 30 min. 85% supernatant may be collected and dialyzed against PBS to remove free DNA (Spectra-Por, Float-A-Lyzer, MWCO=1 MDa). The absorption spectra of the dialyzed solutions may be obtained using a UV-Vis-NIR spectrophotometer (V-780, Jasco). The absorbance at (6,5) E₁₁ may be used to estimate the relative OCC-DNA concentration.

Near-Infrared Hyperspectral Fluorescence Microscopy. Near-infrared fluorescence microscopy may be used to acquire the fluorescence emission of the nanosensors. The system may comprise a continuous wave 730 nm diode laser with an output power of 2 W injected into a multimode fiber to excite the sensors. To ensure a homogeneous illumination over the entire microscope field of view, the excitation beam may be passed through a beam-shaping module to produce a top-hat intensity profile with under 10% power variation on the imaged region of the sample. The power output at the sample stage may be 425.8, 370.2, and 164.8 mW for ×20, ×50, and ×100 objectives, respectively.

A long pass dichroic mirror with a cut-on wavelength of 875 nm (Semrock) may be aligned to reflect the laser to the sample stage of an Olympus IX-71 inverted microscope (with internal optics modified to improve near-infrared transmission from 900 to 1400 nm) equipped with LCPLN20XIR, LCPLN50XIR, and LCPLN100XIR IR objectives (Olympus, USA). Emission may be collected with a thermoelectrically cooled 2D InGaAs array detector (ZephIR 1.7, Photon Etc.). Hyperspectral microscopy may be conducted by passing the emission through a volume Bragg grating placed immediately before the InGaAs array in the optical path. The filtered image produced on the InGaAs camera may be composed of a series of vertical lines, each with a specific wavelength. The reconstruction of a spatially rectified image stack may be performed using cubic interpolation on every pixel for each monochromatic image, according to the wavelength calibration parameters. The rectification may produce a hyperspectral “cube” of images of the same spatial region exhibiting distinct spectral regions with 3.7 nm full width at half maximum. Custom codes, written using MATLAB software, may be used to subtract background, correct for nonuniformities in excitation profile, correct for wavelength-dependent quantum efficiency by each pixel, and compensate for dead pixels on the detector.

Analysis and Processing of Hyperspectral Data. Hyperspectral data acquired may be saved as a (512×640×76) 16-bit array, where the first two coordinates signify the spatial location of a pixel and the last coordinate is its position in wavelength space. The 76-frame wavelength space may range from 950 to 1250 nm. The cube may be divided into two spectral ranges, 950-1050 nm for E₁₁ and 1130-1180 nm for E₁₁-emission. A peak-finding algorithm may be used to calculate the center wavelengths and intensities of E₁₁ and E¹¹-peaks for a given pixel. A data point may be designated as a peak if its intensity was range/4 greater than the intensity of adjacent pixels. To reduce the spectral drift resulting from the movement of lysosomes during the hyperspectral cube acquisition, 8×8 pixels may be combined into a single pixel and the spectral parameters may be obtained from the averaged emission spectrum. Pixels that failed the peak-finding threshold, primarily due to low intensity above the background, may be removed from the data sets. The remaining pixels may be fit with a Lorentzian function.

Preparation of Nanotubes Labeled with Visible Fluorophores. For high-resolution confocal imaging with SWCNTs, the approach may use Cy5 fluorophores attached-DNA strands to encapsulate SWCNTs (Integrated DNA Technologies, sequence=5′//Cy5/GTGTGTGTGTGTGTGTGTGTGTGTGTGTGT//3′). The modified DNA strands may be noncovalently complexed with (6,5)-SWCNT-N(C₂H₅)₂ via the optimized sonication and centrifugation protocol as described above. The synthesized Cy5-tagged SWCNTs may be characterized by absorption spectroscopy.

Near-Infrared Fluorescence Spectroscopy of OCC-DNAs. Fluorescence emission spectra of OCC-DNAs may be acquired using near-infrared fluorescence spectroscopy comprising a tunable white light source, inverted microscope, and 1D InGaAs NIR detector. The SuperK EXTREME supercontinuum white-light laser source (NKT Photonics) may be used with a VARIA variable bandpass filter accessory, capable of tuning the output 500-825 nm, set to a bandwidth of 20 nm centered at 575 nm. The light path may be shaped and fed into the back of an inverted IX-71 microscope (Olympus), where it passes through a 20× or 50×NIR objective (Olympus) and illuminates the samples in a 96-well clear flat bottom UV-transparent microplate (Corning). Emission from the OCC-DNAs may be collected through the objective and passed through a dichroic mirror (875 nm cutoff, Semrock). The light may be f/#matched to the spectrometer using several lenses and injected into a Shamrock 303i spectrograph (Andor, Oxford Instruments) with a slit width of 100 μm, which dispersed the emission using a 86 g/mm grating with 1.35 μm blaze wavelength. The spectral range may be 723-1694 nm with a resolution of 1.89 nm. The light may be collected by an iDus 1.7 μm InGaAs (Andor, Oxford Instruments) with an exposure time of 0.1-15 seconds. An HL-3-CAL-EXT halogen calibration light source (Ocean Optics) may be used to correct for wavelength-dependent features in the emission intensity arising from the spectrometer, detector, and other optics. A Hg/Ne pencil-style calibration lamp (Newport) may be used to calibrate the spectrometer wavelength. Background subtraction may be conducted using a well in a 96-well plate filled with PBS or 10% FBS depending on the experiment. Following acquisition, the data may be processed with custom codes written in MATLAB that apply the aforementioned spectral corrections and background subtraction and fit the fluorescence emission peaks with Lorentzian functions.

Various operations described herein can be implemented on computer systems having various design features. FIG. 19 shows a simplified block diagram of a representative server system 1900 (e.g., computing devices that analyze emission data) and client computer system 1914 (e.g., light detectors and/or computing devices that receive and present analysis results) usable to implement certain embodiments of the present disclosure. In various embodiments, server system 1900 or similar systems can implement services or servers described herein or portions thereof. Client computer system 1914 or similar systems can implement clients described herein.

Server system 1900 can have a modular design that incorporates a number of modules 1902 (e.g., blades in a blade server embodiment); while two modules 1902 are shown, any number can be provided. Each module 1902 can include processing unit(s) 1904 and local storage 1906.

Processing unit(s) 1904 can include a single processor, which can have one or more cores, or multiple processors. In some embodiments, processing unit(s) 1904 can include a general-purpose primary processor as well as one or more special-purpose co-processors such as graphics processors, digital signal processors, or the like. In some embodiments, some or all processing units 1904 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1904 can execute instructions stored in local storage 1906. Any type of processors in any combination can be included in processing unit(s) 1904.

Local storage 1906 can include volatile storage media (e.g., conventional DRAM, SRAM, SDRAM, or the like) and/or non-volatile storage media (e.g., magnetic or optical disk, flash memory, or the like). Storage media incorporated in local storage 1906 can be fixed, removable or upgradeable as desired. Local storage 1906 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1904 need at runtime. The ROM can store static data and instructions that are needed by processing unit(s) 1904. The permanent storage device can be a non-volatile read-and-write memory device that can store instructions and data even when module 1902 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1906 can store one or more software programs to be executed by processing unit(s) 1904, such as an operating system and/or programs implementing various server functions or computing functions, such as any functions of any components of FIGS. 1 and 12 or any other computing device, computing system, and/or sensor identified in this disclosure.

“Software” refers generally to sequences of instructions that, when executed by processing unit(s) 1904 cause server system 1900 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1904. Software can be implemented as a single program or a collection of separate programs or program modules that interact as desired. From local storage 1906 (or non-local storage described below), processing unit(s) 1904 can retrieve program instructions to execute and data to process in order to execute various operations described above.

In some server systems 1900, multiple modules 1902 can be interconnected via a bus or other interconnect 1908, forming a local area network that supports communication between modules 1902 and other components of server system 1900. Interconnect 1908 can be implemented using various technologies including server racks, hubs, routers, etc.

A wide area network (WAN) interface 1910 can provide data communication capability between the local area network (interconnect 1908) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 1906 is intended to provide working memory for processing unit(s) 1904, providing fast access to programs and/or data to be processed while reducing traffic on interconnect 1908. Storage for larger quantities of data can be provided on the local area network by one or more mass storage subsystems 1912 that can be connected to interconnect 1908. Mass storage subsystem 1912 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1912. In some embodiments, additional data storage resources may be accessible via WAN interface 1910 (potentially with increased latency).

Server system 1900 can operate in response to requests received via WAN interface 1910. For example, one of modules 1902 can implement a supervisory function and assign discrete tasks to other modules 1902 in response to received requests. Conventional work allocation techniques can be used. As requests are processed, results can be returned to the requester via WAN interface 1910. Such operation can generally be automated. Further, in some embodiments, WAN interface 1910 can connect multiple server systems 1900 to each other, providing scalable systems capable of managing high volumes of activity. Conventional or other techniques for managing server systems and server farms (collections of server systems that cooperate) can be used, including dynamic resource allocation and reallocation.

Server system 1900 can interact with various user-owned or user-operated devices via a wide-area network such as the Internet. An example of a user-operated device is shown in FIG. 19 as client computing system 1914. Client computing system 1914 can be implemented, for example, as a consumer device such as a smartphone, other mobile phone, tablet computer, wearable computing device (e.g., smart watch, eyeglasses), desktop computer, laptop computer, and so on.

For example, client computing system 1914 can communicate via WAN interface 1910. Client computing system 1914 can include conventional computer components such as processing unit(s) 1916, storage device 1918, network interface 1920, user input device 1922, and user output device 1924. Client computing system 1914 can be a computing device implemented in a variety of form factors, such as a desktop computer, laptop computer, tablet computer, smartphone, other mobile computing device, wearable computing device, or the like.

Processor 1916 and storage device 1918 can be similar to processing unit(s) 1904 and local storage 1906 described above. Suitable devices can be selected based on the demands to be placed on client computing system 1914; for example, client computing system 1914 can be implemented as a “thin” client with limited processing capability or as a high-powered computing device. Client computing system 1914 can be provisioned with program code executable by processing unit(s) 1916 to enable various interactions with server system 1900 of a message management service such as accessing messages, performing actions on messages, and other interactions described above. Some client computing systems 1914 can also interact with a messaging service independently of the message management service.

Network interface 1920 can provide a connection to a wide area network (e.g., the Internet) to which WAN interface 1910 of server system 1900 is also connected. In various embodiments, network interface 1920 can include a wired interface (e.g., Ethernet) and/or a wireless interface implementing various RF data communication standards such as Wi-Fi, Bluetooth, or cellular data network standards (e.g., 3G, 4G, LTE, 5G, etc.).

User input device 1922 can include any device (or devices) via which a user can provide signals to client computing system 1914; client computing system 1914 can interpret the signals as indicative of particular user requests or information. In various embodiments, user input device 1922 can include any or all of a keyboard, touch pad, touch screen, mouse or other pointing device, scroll wheel, click wheel, dial, button, switch, keypad, microphone, and so on.

User output device 1924 can include any device via which client computing system 1914 can provide information to a user. For example, user output device 1924 can include a display-to-display images generated by or delivered to client computing system 1914. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1924 can be provided in addition to or instead of a display. Examples include indicator lights, speakers, tactile “display” devices, printers, haptic devices (e.g., tactile sensory devices may vibrate at different rates or intensities with varying timing), and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1904 and 1916 can provide various functionality for server system 1900 and client computing system 1914, including any of the functionality described herein as being performed by a server or client, or other functionality associated with message management services.

It will be appreciated that server system 1900 and client computing system 1914 are illustrative and that variations and modifications are possible. Computer systems used in connection with embodiments of the present disclosure can have other capabilities not specifically described here. Further, while server system 1900 and client computing system 1914 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

In various embodiments, the codes may be implemented with the logic of CPU-based programming. Multi-core CPUs may be deemed beneficial if some steps are to be run in parallel, though this is not necessary (nor is multi-node). In some of datasets, all images from one visit may take, for example, approximately 3 gigabytes (GB) of memory, and if there are 3 visits each patient, approximately 10 GB may be allocated to preserve similar performance with respect to computational time for certain embodiments discussed above.

Non-limiting example embodiments are provided here:

Embodiment A: A method comprising: receiving, by a computing system, emission data corresponding to fluorescence spectral responses of nanosensor arrays in contact with a plurality of biological samples collected from a cohort of subjects, the cohort of subjects including subjects with a medical condition and subjects without the medical condition, each nanosensor array comprising a semiconducting single-walled carbon nanotubes (SWCNT) that is (i) covalently functionalized and (ii) encapsulated by a nucleic acid; generating, by the computing system, based on the emission data, a dataset comprising a plurality of spectral feature changes caused by the biological samples, the spectral feature changes corresponding to intensity and wavelength of emissions from the nanosensor array in response to excitation by coherent light from a light source; training, by the computing system, a machine learning model based on the dataset and on clinical data corresponding to the medical condition for each subject in the cohort of subjects, wherein the machine learning model comprises at least one of logistic regression, decision tree, artificial neural networks (ANN), random forest, or support vector machine (SVM), wherein the machine learning model is configured to receive emission data and provide a classification corresponding to the medical condition; and providing, by the computing system, the machine learning model for classification of the medical condition in a patient based on spectral responses of nanosensor arrays in contact with a patient sample, wherein providing the machine learning model comprises at least one of storing the machine learning model in a non-volatile computer-readable storage medium of the computing system or transmitting the machine learning model to a second computing system.

Embodiment B: The method of Embodiment A, wherein the spectral feature changes correspond to a plurality of an intensity of an E₁₁ peak (int), an intensity of an E₁₁-peak (int*), a wavelength of the E₁₁ peak (wl), and a wavelength of the and E₁₁-peak (wl*).

Embodiment C: The method of Embodiment A or Embodiment B, wherein the SWCNTs are functionalized by organic color centers (OCCs).

Embodiment D: The method of any of Embodiments A-C, wherein the OCCs comprise an aryl functional group selected from the group consisting of 4-N,N-diethylamino (-4-N(C₂H₅)₂), 3,4,5-trifluoro (-3,4,5-F₃), or 3-fluoro-4-carboxy (-3-F-4-CO₂H).

Embodiment E: The method of any of Embodiments A-D, wherein the SWCNTs are encapsulated by a single-strand deoxyribonucleic acid (ssDNA).

Embodiment F: The method of any of Embodiments A-E, wherein the ssDNA comprises a sequence selected from a group consisting of CTTC₃TTC, (TAT)₄, or (GT)₁₅.

Embodiment G: The method of any of Embodiments A-F, wherein the nanosensor arrays comprise OCC-functionalized, ssDNA-encapsulated SWCNTs selected from a group consisting of NEt₂*CTTC₃TTC, NEt₂*(TAT)₄, NEt₂*(GT)₁₅, 3F*CTTC₃TTC, 3F*(TAT)₄, 3F*(AT)₁₅, 3F*(GT)₁₅, F—CO₂H*CTTC₃TTC, F—CO₂H*(AT)₁₅, or F—CO₂H*(GT)₁₅, where NEt₂ represents 4-N,N-diethylamino, 3F represents F—CO₂H 3,4,5-trifluoro, and F—CO₂H represents 3-fluoro-4-carboxy aryl OCCs.

Embodiment H: The method of any of Embodiments A-C, wherein the machine learning model is an SVM model trained by spectral responses of a plurality of OCC-DNA SWCNTs.

Embodiment I: The method of any of Embodiments A-H, wherein the plurality of OCC-DNA SWCNTs comprise at least one OCC-DNA SWCNT selected from a group consisting of NEt₂*CTTC₃TTC, NEt₂*(TAT)₄, 3F*(TAT)₄, 3F*(AT)₁₅, or 3F*(GT)₁₅, where NEt₂ represents 4-N,N-diethylamino, 3F represents F—CO₂H 3,4,5-trifluoro, and F—CO₂H represents 3-fluoro-4-carboxy aryl OCCs.

Embodiment J: The method of any of Embodiments A-I, further comprising: receiving, by the computing system, emission data corresponding to fluorescence spectral responses of a nanosensor array in contact with a biological sample of a patient; and processing, by the computing system, the emission data using the machine learning to obtain a classification corresponding to the medical condition in the patient.

Embodiment K: The method of any of Embodiments A-J, the method further comprising administering a treatment to the patient based on the classification.

Embodiment L: The method of any of Embodiments A-K, wherein the biological sample of the patient is a serum sample from the patient.

Embodiment M: The method of any of Embodiments A-L, wherein the coherent light used for excitation has a wavelength bandwidth centered at 575 nanometers (nm).

Embodiment N: The method of any of Embodiments A-M, further comprising synthesizing the nanosensor arrays.

Embodiment O: The method of any of Embodiments A-N, wherein synthesizing the nanosensor arrays comprises introducing sp³ defects to (6,5) SWCNTs via diazonium chemistry and encapsulating the SWCNTs with a library ssDNA to solubilize the nanosensors in biofluids.

Embodiment P: The method of any of Embodiments A-O, wherein the biological samples comprise sera of subjects in the cohort of subjects.

Embodiment Q: A method comprising: receiving, by a computing system, emission data corresponding to fluorescence spectral responses of a nanosensor array in contact with a biological sample of the patient, the nanosensor array comprising a semiconducting single-walled carbon nanotubes (SWCNT) that is (i) covalently functionalized and (ii) encapsulated by a nucleic acid; and processing, by the computing system, the emission data using a machine learning model to obtain a classification corresponding to a medical condition in the patient, the machine learning model configured to provide the classification based on emission data corresponding to biological sample of the patient, the machine learning model having been trained based on reference emission data and clinical data corresponding to the medical condition for each subject in a cohort of subjects, the reference emission data corresponding to fluorescence spectral responses of nanosensor arrays in contact with a plurality of biological samples collected from the cohort of subjects, the cohort of subjects including subjects with a medical condition and subjects without the medical condition, the emission data having been used to generate a training dataset comprising a plurality of spectral feature changes caused by the biological samples, the spectral feature changes corresponding to intensity and wavelength of emissions from the nanosensor array in response to excitation by coherent light from a light source, wherein the machine learning model comprises at least one of logistic regression, decision tree, artificial neural networks (ANN), random forest, or support vector machine (SVM).

Embodiment R: The method of Embodiment Q, the method further comprising administering a treatment to the patient based on the classification.

Embodiment S: The method of Embodiment Q or R, wherein the SWCNTs are functionalized by organic color centers (OCCs), and the SWCNTs are encapsulated by a single-strand deoxyribonucleic acid (ssDNA).

Embodiment T: The method of any of Embodiments Q-S The method of claim [0154]19, wherein the OCCs comprise an aryl functional group selected from the group consisting of 4-N,N-diethylamino (-4-N(C₂H₅)₂), 3,4,5-trifluoro (-3,4,5-F₃), or 3-fluoro-4-carboxy (-3-F-4-CO₂H), and the ssDNA comprises a sequence selected from a group consisting of CTTC₃TTC, (TAT)₄, or (GT)₁₅.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

Additional background and supporting information can be found in the following documents, each of which is herein incorporated by reference:

### PRIMARY REFERENCES

1. Bray, F. et al. Global cancer statistics 2018: GLOBOCAN estimates of incidence and mortality worldwide for 36 cancers in 185 countries. CA Cancer J. Clin. 68, 394-424 (2018).
2. Siegel, R. L., Miller, K. D. & Jemal, A. Cancer statistics, 2020. CA Cancer J. Clin. 70, 7-30 (2020).
3. Menon, U. et al. Risk Algorithm Using Serial Biomarker Measurements Doubles the Number of Screen-Detected Cancers Compared With a Single-Threshold Rule in the United Kingdom Collaborative Trial of Ovarian Cancer Screening. J. Clin. Oncol. 33, 2062-2071 (2015).
4. Blyuss, O. et al. Comparison of Longitudinal CA125 Algorithms as a First-Line Screen for Ovarian Cancer in the General Population. Clin. Cancer Res. 24, 4726 (2018).
5. Jacobs, I. J. et al. Ovarian cancer screening and mortality in the UK Collaborative Trial of Ovarian Cancer Screening (UKCTOCS): a randomised controlled trial. Lancet 387, 945-956 (2016).
6. Menon, U. et al. Ovarian cancer population screening and mortality after long-term follow-up in the UK Collaborative Trial of Ovarian Cancer Screening (UKCTOCS): a randomised controlled trial. Lancet 397, 2182-2193 (2021).
7. Dupont, J. et al. Early Detection and Prognosis of Ovarian Cancer Using Serum YKL-40. J. Clin. Oncol. 22, 3330-3339 (2004).
8. Cramer, D. W. et al. Ovarian Cancer Biomarker Performance in Prostate, Lung, Colorectal, and Ovarian Cancer Screening Trial Specimens. Cancer Prev. Res. 4, 365 (2011).
9. Han, C. et al. A novel multiple biomarker panel for the early detection of high-grade serous ovarian carcinoma. Gynecol. Oncol. 149, 585-591 (2018).
10. Moore, L. E. et al. Proteomic biomarkers in combination with CA 125 for detection of epithelial ovarian cancer using prediagnostic serum samples from the Prostate, Lung, Colorectal, and Ovarian (PLCO) Cancer Screening Trial. Cancer 118, 91-100 (2012).
11. Johnson, C. C. et al. The epidemiology of CA-125 in women without evidence of ovarian cancer in the Prostate, Lung, Colorectal and Ovarian Cancer (PLCO) Screening Trial. Gynecol. Oncol. 110, 383-389 (2008).
12. Bast, R. C. et al. A Radioimmunoassay Using a Monoclonal Antibody to Monitor the Course of Epithelial Ovarian Cancer. N. Engl. J. Med. 309, 883-887 (1983).
13. Miralles, C. et al. Cancer antigen 125 associated with multiple benign and malignant pathologies. Ann. Surg. Oncol. 10, 150-154 (2003).
14. Buamah, P. Benign conditions associated with raised serum CA-125 concentration. J. Surg. Oncol. 75, 264-265 (2000).
15. Linda, H. et al. Human epididymis protein 4 (HE4) in benign and malignant diseases. Clin. Chem. Lab. Med. 50, 2181-2188 (2012).
16. Moss, E. L., Hollingworth, J. & Reynolds, T. M. The role of CA125 in clinical practice. J. Clin. Pathol. 58, 308 (2005).
17. Park, Y., Lee, J.-H., Hong, D. J., Lee, E. Y. & Kim, H.-S. Diagnostic performances of HE4 and CA125 for the detection of ovarian cancer from patients with various gynecologic and non-gynecologic diseases. Clin. Biochem. 44, 884-888 (2011).
18. Diamandis, E. P. The failure of protein cancer biomarkers to reach the clinic: why, and what can be done to address the problem? BMC Med. 10, 87 (2012).
19. Su, C.-Y., Menuz, K. & Carlson, J. R. Olfactory perception: Receptors, cells, and circuits. Cell 139, 45-59 (2009).
20. Liu, M. C. et al. Sensitive and specific multi-cancer detection and localization using methylation signatures in cell-free DNA. Ann. Oncol. 31, 745-759 (2020).
21. Hao, Y. et al. in SENSORS, 2003 IEEE, Vol. 2 1333-1337 Vol. 1332 (2003).
22. Zhang, J. et al. Nondestructive tissue analysis for ex vivo and in vivo cancer diagnosis using a handheld mass spectrometry system. Sci. Transl. Med. 9, eaan3968 (2017).
23. Yu, K.-H. et al. Predicting non-small cell lung cancer prognosis by fully automated microscopic pathology image features. Nat. Commun. 7, 12474 (2016).
24. Bera, K., Schalper, K. A., Rimm, D. L., Velcheti, V. & Madabhushi, A. Artificial intelligence in digital pathology new tools for diagnosis and precision oncology. Nat. Rev. Clin. Oncol. 16, 703-715 (2019).
25. Rajkomar, A. et al. Scalable and accurate deep learning with electronic health records. npj Digit. Med. 1, 18 (2018).
26. Bachilo, S. M. et al. Structure-assigned optical spectra of single-walled carbon nanotubes. Science 298, 2361 (2002).
27. Cognet, L. et al. Stepwise quenching of exciton fluorescence in carbon nanotubes by single-molecule reactions. Science 316, 1465 (2007).
28. Heller, D. A. et al. Optical detection of DNA conformational polymorphism on single-walled carbon nanotubes. Science 311, 508 (2006).
29. Jena, P. V. et al. A carbon nanotube optical reporter maps endolysosomal lipid flux. ACS Nano 11, 10689-10703 (2017).
30. Heller, D. A. et al. Multimodal optical sensing and analyte specificity using single-walled carbon nanotubes. Nat. Nanotech. 4, 114-120 (2009).
31. Roxbury, D., Jena, P. V., Shamay, Y., Horoszko, C. P. & Heller, D. A. Cell membrane proteins modulate the carbon nanotube optical bandgap via surface charge accumulation. ACS Nano 10, 499-506 (2016).
32. Williams, R. M. et al. Noninvasive ovarian cancer biomarker detection via an optical nanosensor implant. Sci. Adv. 4, eaaq1090 (2018).
33. Roxbury, D., Mittal, J. & Jagota, A. Molecular-basis of single-walled carbon nanotube recognition by single-stranded DNA. Nano Lett. 12, 1464-1469 (2012).
34. Roxbury, D., Jagota, A. & Mittal, J. Structural characteristics of oligomeric DNA strands adsorbed onto single-walled carbon nanotubes. J. Phys. Chem. B 117, 132-140 (2013).
35. Roxbury, D., Tu, X., Zheng, M. & Jagota, A. Recognition ability of DNA for carbon nanotubes correlates with their binding affinity. Langmuir 27, 8282-8293 (2011).
36. Horoszko, C. P., Jena, P. V., Roxbury, D., Rotkin, S. V. & Heller, D. A. Optical voltammetry of polymer-encapsulated single-walled carbon nanotubes. J. Phys. Chem. C 123, 24200-24208 (2019).
37. Brozena, A. H., Kim, M., Powell, L. R. & Wang, Y. Controlling the optical properties of carbon nanotubes with organic colour-centre quantum defects. Nat. Rev. Chem.
3, 375-392 (2019).
38. Kwon, H. et al. Optical probing of local pH and temperature in complex fluids with covalently functionalized, semiconducting carbon nanotubes. J. Phys. Chem. C 119, 3733-3739 (2015).
39. Luo, H.-B. et al. One-pot, large-scale synthesis of organic color center-tailored semiconducting carbon nanotubes. ACS Nano 13, 8417-8424 (2019).
40. Ao, G., Streit, J. K., Fagan, J. A. & Zheng, M. Differentiating left- and right-handed carbon nanotubes by DNA. J. Am. Chem. Soc. 138, 16677-16685 (2016).
41. Shahriari, B., Swersky, K., Wang, Z., Adams, R. P. & De Freitas, N. Taking the human out of the loop: A review of Bayesian optimization. Proc. IEEE 104, 148-175 (2016).
42. Wolff, R. F. et al. PROBAST: A Tool to Assess the Risk of Bias and Applicability of Prediction Model Studies. Ann. Intern. Med. 170, 51-58 (2019).
43. Pinals, R. L., Yang, D., Lui, A., Cao, W. & Landry, M. P. Corona Exchange Dynamics on Carbon Nanotubes by Multiplexed Fluorescence Monitoring. J. Am. Chem. Soc. 142, 1254-1264 (2020).
44. Tenzer, S. et al. Rapid formation of plasma protein corona critically affects nanoparticle pathophysiology. Nat. Nanotech. 8, 772-781 (2013).
45. Heller, D. A. et al. Peptide secondary structure modulates single-walled carbon nanotube fluorescence as a chaperone sensor for nitroaromatics. Proc. Natl. Acad. Sci. U.S.A. 108, 8544 (2011).
46. Wu, X., Kim, M., Qu, H. & Wang, Y. Single-defect spectroscopy in the shortwave infrared. Nat. Commun. 10, 2672 (2019).
47. Lee, M. A. et al. Can Fish and Cell Phones Teach Us about Our Health? ACS Sens. 4, 2566-2570 (2019).
48. Zednik, C. Solving the Black Box Problem: A Normative Framework for Explainable Artificial Intelligence. Philos. Technol. 34, 265-288 (2021).
49. Docter, D. et al. Quantitative profiling of the protein coronas that form around nanoparticles. Nat. Protoc. 9, 2030-2044 (2014).
50. Pinals, R. L. et al. Quantitative Protein Corona Composition and Dynamics on Carbon Nanotubes in Biological Environments. Angew. Chem. Int. Ed. 59, 23668-23677 (2020).
51. Lai, Z. W., Yan, Y., Caruso, F. & Nice, E. C. Emerging Techniques in Proteomics for Probing Nano-Bio Interactions. ACS Nano 6, 10438-10448 (2012).
52. Hadjidemetriou, M. et al. Nano-scavengers for blood biomarker discovery in ovarian carcinoma. Nano Today 34, 100901 (2020).
53. Zheng, M. & Diner, B. A. Solution redox chemistry of carbon nanotubes. J. Am. Chem. Soc. 126, 15490-15494 (2004).
54. Chawla, N. V., Bowyer, K. W., Hall, L. O. & Kegelmeyer, W. P. SMOTE: Synthetic Minority Over-Sampling Technique. J. Artif Intell. Res. 16, 321-357 (2002).
55. Pedregosa, F. et al. Scikit-learn: Machine learning in Python. J. Mach. Learn. Res. 12, 2825-2830 (2011).

### ADDITIONAL REFERENCES

1. Lawrence, R. E. & Zoncu, R. The lysosome as a cellular centre for signalling, metabolism and quality control. Nat. Cell Biol. 21, 133-142 (2019).
2. Mulcahy Levy, J. M. & Thorburn, A. Autophagy in cancer: moving from understanding mechanism to improving therapy responses in patients. Cell Death Differ. 27, 843-857 (2020).
3. Zhan, L. et al. Autophagy as an emerging therapy target for ovarian carcinoma. Oncotarget 7, 83476-83487 (2016).
4. Jung, S., Jeong, H. & Yu, S.-W. Autophagy as a decisive process for cell death. Exp. Mol. Med. 52, 921-930 (2020).
5. Nakadera, E. et al. Inhibition of mTOR improves the impairment of acidification in autophagic vesicles caused by hepatic steatosis. Biochem. Biophys. Res. Commun. 469, 1104-1110 (2016).
6. Azoulay-Alfaguter, I., Elya, R., Avrahami, L., Katz, A. & Eldar-Finkelman, H. Combined regulation of mTORC1 and lysosomal acidification by GSK-3 suppresses autophagy and contributes to cancer cell growth. Oncogene 34, 4613-4623 (2015).
7. Williams, R. M. et al. Harnessing nanotechnology to expand the toolbox of chemical biology. Nat. Chem. Biol. 17, 129-137 (2021).
8. Guha, S. et al. Approaches for detecting lysosomal alkalinization and impaired degradation in fresh and cultured RPE cells: Evidence for a role in retinal degenerations. Exp. Eye Res. 126, 68-76 (2014).
9. Ma, L., Ouyang, Q., Werthmann, G. C., Thompson, H. M. & Morrow, E. M. Live-cell microscopy and fluorescence-based measurement of luminal pH in intracellular organelles. Front. Cell Dev. Biol. 5, 71 (2017).
10. Bachilo, S. M. et al. Structure-assigned optical spectra of single-walled carbon nanotubes. Science 298, 2361 (2002).
11. Welsher, K., Sherlock, S. P. & Dai, H. Deep-tissue anatomical imaging of mice using carbon nanotube fluorophores in the second near-infrared window. Proc. Natl. Acad. Sci. U.S.A. 108, 8943 (2011).
12. Mandal, A. K. et al. Fluorescent sp3 defect-tailored carbon nanotubes enable NIR-II single particle imaging in live brain slices at ultra-low excitation doses. Sci. Rep. 10, 5286 (2020).
13. Jena, P. V. et al. A carbon nanotube optical reporter maps endolysosomal lipid flux. ACS Nano 11, 10689-10703 (2017).
14. Galassi, T. V. et al. An optical nanoreporter of endolysosomal lipid accumulation reveals enduring effects of diet on hepatic macrophages in vivo. Sci. Transl. Med. 10, eaar2680 (2018).
15. Galassi, T. V. et al. Long-term in vivo biocompatibility of single-walled carbon nanotubes. PLOS ONE 15, e0226791 (2020).
16. Brozena, A. H., Kim, M., Powell, L. R. & Wang, Y. Controlling the optical properties of carbon nanotubes with organic colour-centre quantum defects. Nat. Rev. Chem. 3, 375-392 (2019).
17. Kwon, H. et al. Optical probing of local pH and temperature in complex fluids with covalently functionalized, semiconducting carbon nanotubes. J. Phys. Chem. C 119, 3733-3739 (2015).
18. Piao, Y. et al. Brightening of carbon nanotube photoluminescence through the incorporation of sp3 defects. Nat. Chem. 5, 840-845 (2013).
19. Gravely, M., Safaee, M. M. & Roxbury, D. Biomolecular functionalization of a nanomaterial to control stability and retention within live cells. Nano Lett. 19, 6203-6212 (2019).
20. Jena, P. V., Safaee, M. M., Heller, D. A. & Roxbury, D. DNA-carbon nanotube complexation affinity and photoluminescence modulation are independent. ACS Appl. Mater. Interfaces 9, 21397-21405 (2017).
21. Miyauchi, Y. et al. Dependence of exciton transition energy of single-walled carbon nanotubes on surrounding dielectric materials. Chem. Phys. Lett. 442, 394-399 (2007).
22. Pinals, R. L., Yang, D., Lui, A., Cao, W. & Landry, M. P. Corona Exchange Dynamics on Carbon Nanotubes by Multiplexed Fluorescence Monitoring. J. Am. Chem. Soc. 142, 1254-1264 (2020).
23. Roxbury, D. et al. Hyperspectral microscopy of near-infrared fluorescence enables 17-chirality carbon nanotube imaging. Sci. Rep. 5, 14167 (2015).
24. Yamamoto, A. et al. Bafilomycin A1 prevents maturation of autophagic vacuoles by inhibiting fusion between autophagosomes and lysosomes in rat hepatoma cell Line, H-4-II-E cells. Cell Struct. Funct. 23, 33-42 (1998).
25. Mauvezin, C. & Neufeld, T. P. Bafilomycin A1 disrupts autophagic flux by inhibiting both V-ATPase-dependent acidification and Ca-P60A/SERCA-dependent autophagosome-lysosome fusion. Autophagy 11, 1437-1438 (2015).
26. Chung, C. Y.-S. et al. Covalent targeting of the vacuolar H+-ATPase activates autophagy via mTORC1 inhibition. Nat. Chem. Biol. 15, 776-785 (2019).
27. Pena-Llopis, S. et al. Regulation of TFEB and V-ATPases by mTORC1. EMBO J. 30, 3242-3258 (2011).
28. Settembre, C. et al. TFEB links autophagy to lysosomal biogenesis. Science 332, 1429 (2011).
29. Thoreen, C. C. et al. An ATP-competitive mammalian target of Rapamycin inhibitor reveals Rapamycin-resistant functions of mTORC1*. J. Biol. Chem. 284, 8023-8032 (2009).
30. Yim, W. W.-Y. & Mizushima, N. Lysosome biology in autophagy. Cell Discov. 6, 6 (2020).
31. Mizushima, N. & Murphy, L. O. Autophagy assays for biological discovery and therapeutic development. Trends Biochem. Sci. 45, 1080-1093 (2020).
32. Klionsky, D. J. et al. Guidelines for the use and interpretation of assays for monitoring autophagy (4th edition). Autophagy 17, 1-382 (2021).
33. Sahani, M. H., Itakura, E. & Mizushima, N. Expression of the autophagy substrate SQSTM1/p62 is restored during prolonged starvation depending on transcriptional upregulation and autophagy-derived amino acids. Autophagy 10, 431-441 (2014).
34. Roxbury, D., Jena, P. V., Shamay, Y., Horoszko, C. P. & Heller, D. A. Cell membrane proteins modulate the carbon nanotube optical bandgap via surface charge accumulation. ACS Nano 10, 499-506 (2016).
35. Harvey, J. D. et al. A carbon nanotube reporter of microRNA hybridization events in vivo. Nat. Biomed. Eng. 1, 0041 (2017).
36. Quintero, B., Cabeza, M. C., Martinez, M. I., Gutierrez, P. & Martinez, P. J. Dediazoniation of p-hydroxy and p-nitrobenzenediazonium ions in an aqueous medium: Interference by the chelating agent diethylenetriaminepentaacetic acid. Can. J. Chem. 81, 832-839 (2003).
37. Streit, J. K., Fagan, J. A. & Zheng, M. A low energy route to DNA-wrapped carbon nanotubes via replacement of bile salt surfactants. Anal. Chem. 89, 10496-10503 (2017).
38. Bankhead, P. et al. QuPath: Open source software for digital pathology image analysis. Sci. Rep. 7, 16878 (2017).

What is claimed is:

1. A method comprising:
receiving, by a computing system, emission data corresponding to fluorescence spectral responses of nanosensor arrays in contact with a plurality of biological samples collected from a cohort of subjects, the cohort of subjects including subjects with a medical condition and subjects without the medical condition, each nanosensor array comprising a semiconducting single-walled carbon nanotubes (SWCNT) that is (i) covalently functionalized and (ii) encapsulated by a nucleic acid;
generating, by the computing system, based on the emission data, a dataset comprising a plurality of spectral feature changes caused by the biological samples, the spectral feature changes corresponding to intensity and wavelength of emissions from the nanosensor array in response to excitation by coherent light from a light source;
training, by the computing system, a machine learning model based on the dataset and on clinical data corresponding to the medical condition for each subject in the cohort of subjects, wherein the machine learning model comprises at least one of logistic regression, decision tree, artificial neural networks (ANN), random forest, or support vector machine (SVM), wherein the machine learning model is configured to receive emission data and provide a classification corresponding to the medical condition; and
providing, by the computing system, the machine learning model for classification of the medical condition in one or more patients based on spectral responses of nanosensor arrays in contact with one or more patient samples, wherein providing the machine learning model comprises at least one of storing the machine learning model in a non-volatile computer-readable storage medium of the computing system or transmitting the machine learning model to a second computing system.

2. The method of claim 1, wherein the spectral feature changes correspond to a plurality of an intensity of an E₁₁ peak (int), an intensity of an E₁₁-peak (int*), a wavelength of the E₁₁ peak (wl), and a wavelength of the and E₁₁-peak (wl*).

3. The method of claim 1, wherein the SWCNTs are functionalized by organic color centers (OCCs).

4. The method of claim 3, wherein the OCCs comprise an aryl functional group selected from the group consisting of 4-N,N-diethylamino (-4-N(C₂H₅)₂), 3,4,5-trifluoro (-3,4,5-F₃), or 3-fluoro-4-carboxy (-3-F-4-CO₂H).

5. The method of claim 1, wherein the SWCNTs are encapsulated by a single-strand deoxyribonucleic acid (ssDNA).

6. The method of claim 5, wherein the ssDNA comprises a sequence selected from a group consisting of CTTC₃TTC, (TAT)₄, or (GT)₁₅.

7. The method of claim 1, wherein the nanosensor arrays comprise OCC-functionalized, ssDNA-encapsulated SWCNTs selected from a group consisting of NEt₂*CTTC₃TTC, NEt₂*(TAT)₄, NEt₂*(GT)₁₅, 3F*CTTC₃TTC, 3F*(TAT)₄, 3F*(AT)₁₅, 3F*(GT)₁₅, F—CO₂H*CTTC₃TTC, F—CO₂H*(AT)₁₅, or F—CO₂H*(GT)₁₅, where NEt₂ represents 4-N,N-diethylamino, 3F represents F—CO₂H 3,4,5-trifluoro, and F—CO₂H represents 3-fluoro-4-carboxy aryl OCCs.

8. The method of claim 1, wherein the machine learning model is an SVM model trained by spectral responses of a plurality of OCC-DNA SWCNTs.

9. The method of claim 8, wherein the plurality of OCC-DNA SWCNTs comprise at least one OCC-DNA SWCNT selected from a group consisting of NEt₂*CTTC₃TTC, NEt₂*(TAT)₄, 3F*(TAT)₄, 3F*(AT)₁₅, or 3F*(GT)₁₅, where NEt₂ represents 4-N,N-diethylamino, 3F represents F—CO₂H 3,4,5-trifluoro, and F—CO₂H represents 3-fluoro-4-carboxy aryl OCCs.

10. The method of claim 1, further comprising:
receiving, by the computing system, emission data corresponding to fluorescence spectral responses of a nanosensor array in contact with a biological sample of a patient; and
processing, by the computing system, the emission data using the machine learning model to obtain a classification corresponding to the medical condition in the patient.

11. The method of claim 10, the method further comprising administering a treatment to the patient based on the classification.

12. The method of claim 10, wherein the biological sample of the patient is a serum sample from the patient.

13. The method of claim 1, wherein the coherent light used for excitation has a wavelength bandwidth centered at 575 nanometers (nm).

14. The method of claim 1, further comprising synthesizing the nanosensor arrays.

15. The method of claim 14, wherein synthesizing the nanosensor arrays comprises introducing sp³ defects to (6,5) SWCNTs via diazonium chemistry and encapsulating the SWCNTs with a library ssDNA to solubilize the nanosensors in biofluids.

16. The method of claim 1, wherein the biological samples comprise sera of subjects in the cohort of subjects.

17. A method comprising:
receiving, by a computing system, emission data corresponding to fluorescence spectral responses of a nanosensor array in contact with a biological sample of the patient, the nanosensor array comprising a semiconducting single-walled carbon nanotubes (SWCNT) that is (i) covalently functionalized and (ii) encapsulated by a nucleic acid; and
processing, by the computing system, the emission data using a machine learning model to obtain a classification corresponding to a medical condition in the patient, the machine learning model configured to provide the classification based on emission data corresponding to biological sample of the patient, the machine learning model having been trained based on reference emission data and clinical data corresponding to the medical condition for each subject in a cohort of subjects, the reference emission data corresponding to fluorescence spectral responses of nanosensor arrays in contact with a plurality of biological samples collected from the cohort of subjects, the cohort of subjects including subjects with a medical condition and subjects without the medical condition, the emission data having been used to generate a training dataset comprising a plurality of spectral feature changes caused by the biological samples, the spectral feature changes corresponding to intensity and wavelength of emissions from the nanosensor array in response to excitation by coherent light from a light source, wherein the machine learning model comprises at least one of logistic regression, decision tree, artificial neural networks (ANN), random forest, or support vector machine (SVM).

18. The method of claim 17, the method further comprising administering a treatment to the patient based on the classification.

19. The method of claim 17, wherein the SWCNTs are functionalized by organic color centers (OCCs), and the SWCNTs are encapsulated by a single-strand deoxyribonucleic acid (ssDNA).

20. The method of claim 19, wherein the OCCs comprise an aryl functional group selected from the group consisting of 4-N,N-diethylamino (-4-N(C₂H₅)₂), 3,4,5-trifluoro (-3,4,5-F₃), or 3-fluoro-4-carboxy (-3-F-4-CO₂H), and the ssDNA comprises a sequence selected from a group consisting of CTTC₃TTC, (TAT)₄, or (GT)₁₅.
